(19) United States
(12) Patent Application Publication
(10) Pub. No.: US 2026/0248825 A1
(43) Pub. Date: Aug. 27, 2026

(54) ORAL DOSAGE FORMS FOR THE TREATMENT OF LIVER DISORDERS AND METHODS OF PREPARING THE SAME

(71) Applicant: VIKING THERAPEUTICS, INC., San Diego, CA (US)

(72) Inventors: Geoffrey E. Barker, Carlsbad, CA (US); Maureen Barnes, San Diego, CA (US); William Hoye, San Diego, CA (US)

(21) Appl. No.: 19/130,526

(22) PCT Filed: Nov. 15, 2023
(86) PCT No.: PCT/US2023/079852

§ 371 Date: May 15, 2025

Related U.S. Application Data
(60) Provisional application No. 63/426,161, filed on Nov. 17, 2022.

(51) Int. Cl. A61K 31/665; A61K 9/00; A61K 9/48

(52) U.S. Cl. CPC A61K 31/665; A61K 9/0053; A61K 9/4858; A61K 9/4866

(57) ABSTRACT

The present disclosure is directed to oral forms of TRβ agonists and methods of preparing the same. The disclosure provides oral dosage forms of TRβ agonists with improved stability properties that allow for storage under normal conditions.

### CROSS-REFERENCE TO RELATED APPLICATIONS

This application claims priority to U.S. Prov. Patent App. No. 63/426,161, titled “ORAL DOSAGE FORMS FOR THE TREATMENT OF LIVER DISORDERS AND METHODS OF PREPARING THE SAME” and filed on Nov. 17, 2022, the disclosure of which is hereby incorporated herein by reference in its entirety.

### FIELD

The compositions and methods of the present disclosure relate generally to the field of oral dosage forms of thyroid hormone receptor-β (TRβ) agonists for the treatment of liver disorders, fibrotic disease and inflammation.

### BACKGROUND

Thyroid hormones (TH) are synthesized in the thyroid in response to thyroid stimulating hormone (TSH), which is secreted by the pituitary gland in response to various stimulants (e.g., thyrotropin-releasing hormone (TRH) from the hypothalamus). Thyroid hormones are iodinated O-aryl tyrosine analogues excreted into the circulation primarily as 3,3′,5,5′-tetraiodothyronine (T4). T4 is rapidly deiodinated in local tissues by thyroxine 5′-deiodinase to 3,3′,5′-triiodothyronine (T3), which is the most potent TH. T3 is metabolized to inactive metabolites via a variety of pathways, including pathways involving deiodination, glucuronidation, sulfation, deamination, and decarboxylation. Most of the circulating T4 and T3 is eliminated through the liver.

The biological activity of THs is mediated largely through thyroid hormone receptors (TRs). TRs belong to the nuclear receptor superfamily, which, along with its common partner, the retinoid X receptor, form heterodimers that act as ligand-inducible transcription factors. Like other nuclear receptors, TRs have a ligand binding domain and a DNA binding domain and regulate gene expression through ligand-dependent interactions with DNA response elements (thyroid response elements, TREs). Currently, the literature shows that TRs are encoded by two distinct genes (TRα and TRβ), which produce several isoforms through alternative splicing (Williams, Mol. Cell. Biol. 20 (22): 8329-42 (2000); Nagaya et al., Biochem. Biophys. Res. Commun. 226 (2): 426-30 (1996)). The major isoforms that have so far been identified are TRα-1, TRα-2, TRβ-1 and TRβ-2. TRα-1 is ubiquitously expressed in the rat with highest expression in skeletal muscle and brown fat. TRβ-1 is also ubiquitously expressed with highest expression in the liver, brain and kidney. TRβ-2 is expressed in the anterior pituitary gland and specific regions of the hypothalamus as well as the developing brain and inner ear. In the rat and mouse liver, TRβ-1 is the predominant isoform (80%). The TR isoforms found in human and rat are highly homologous with respect to their amino acid sequences which suggest that each serves a specialized function.

TH's affect the growth, metabolism and the physiological function of nearly all organs. TH's lower serum cholesterol and triglycerides. However, side effects of TH action include cardiac arrhythmia, bone loss, nervousness, and anxiety.

TRβ agonists may be useful as therapeutics for conditions such as hepatitis, non-alcoholic fatty liver disease, non-alcoholic steatohepatitis (NASH), and a variety of fibrotic disease and disorder. However, some TRβ agonists may be poorly soluble and/or have an undesirable stability profiles. Low aqueous solubility and poor stability of such compounds may require special storage conditions of compounds and may present challenges in preparing formulations that provide adequate exposure to a subject and that do not degrade when stored under normal conditions.

Accordingly, a need exists to provide drug formulations that provide suitable exposure of a drug with poor aqueous solubility to a subject. Furthermore, a need exists for drug formulations with improved stability such that the drug does not degrade under ambient conditions and room temperature storage can be utilized.

### SUMMARY

A first aspect of the present disclosure relates to an oral dosage form comprising a composition comprising: Compound 1 having the structure:

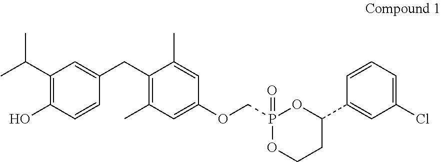

or a pharmaceutically acceptable salt thereof; and one or more liquid excipients selected from the group consisting of polyethylene glycol, caprylic acid triglyceride, capric acid triglyceride, caprylic acid diiglycerides, capric acid diglycerides, caprylic acid monoglycerides, capric acid monoglycerides, polyethylene glycol sorbitan monooleate, oleic acid, polyethoxylated castor oil, lauroyl polyoxyl-32 glycerides, sorbitan monooleate, phosphatidylcholine, glyceryl stearate, oleaic acid, ascorbyl palmitate, lecithin, sorbitan monolaurate, tocophersolan, ethanol, propylene glycol monocaprylate, linoleic acid monoglyceride, oleic acid monoglyceride, linoleic acid diglyceride, oleic acid diglyceride, linoleic acid triglyceride, oleic acid triglyceride, glycerol monocaprylocaprate, and combinations thereof.

In some embodiments of the first aspect, the composition further comprises an antioxidant. In some embodiments, the antioxidant is butylated hydroxytoluene.

In some embodiments of the first aspect, the oral dosage form described herein is in the form of a gelatin-based capsule.

In some embodiments of the first aspect, Compound 1 is present in the composition at a concentration of from 0.1 mg to 100 mg per mL of excipient.

In some embodiments of the first aspect, the excipient comprises Maisine® CC (glycerol/glyceryl monolinoleate). In some embodiments, the excipient comprises 20% to 60% Maisine® CC by weight.

In some embodiments of the first aspect, the oral dosage form described herein may be characterized by having from 95% to 100% of the original amount of Compound 1 after 1 month of storage at 25° C. and 60% relative humidity (RH). In other embodiments, the oral dosage form described herein may be characterized by having from 98% to 100% of the original amount of Compound 1 after 1 month of storage at 25° C. and 60% relative humidity (RH). In some embodiments, the oral dosage form described herein may be characterized by having from 95% to 100% of the original amount of Compound 1 after 2 months of storage at 25° C. and 60% relative humidity (RH). In other embodiments, the oral dosage form described herein may be characterized by having from 98% to 100% of the original amount of Compound 1 after 2 months of storage at 25° C. and 60% relative humidity (RH).

A second aspect of the present disclosure relates to a method of preventing, treating, or ameliorating one or more fatty liver diseases in a subject in need thereof comprising administering to said subject in need thereof, the method comprising administering the oral dosage described herein.

In some embodiments of the second aspect, the fatty liver disease is selected from the group consisting of steatosis, non-alcoholic fatty liver disease, and non-alcoholic steatohepatitis.

In some embodiments of the second aspect, wherein the method comprises administration of a second pharmaceutical agent. In some embodiments, the second pharmaceutical agent may be administered sequentially or simultaneously.

In some embodiments of the second aspect, the method may result in the prevention, treatment, or amelioration, of a fibrosis, fibrotic condition, or fibrotic symptom. In some embodiments, the method may result in the reduction in the amount of extracellular matrix proteins present in one or more tissues of said subject. In some embodiments, the method may result in the reduction in the amount of collagen present in one or more tissues of said subject. In some embodiments, the method may result in the reduction in the amount of Type I, Type Ia, or Type III collagen present in one or more tissues of said subject.

### DETAILED DESCRIPTION

The present disclosure provides oral dosage formulations of Compound 1:

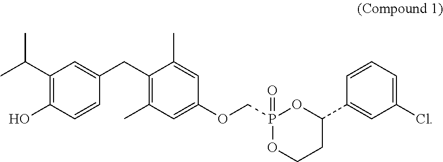

Compound 1 is a low solubility, lipophilic prodrug compound in development for the treatment of chronic liver diseases including non-alcoholic fatty liver disease (NAFLD). Compound 1 may be prepared according to known methods, including those described in U.S. Pat. No. 7,829,552, which is incorporated herein by reference in its entirety. The current clinical dosage form is an encapsulated PEG-based formulation that provides adequate exposure but requires cold chain storage due to chemical stability challenges. Provided herein are softgel formulations (i.e., formulations in a gelatin-based shell) for oral administration that are sufficiently effective for oral administration to a subject and sufficiently stable for storage under normal conditions.

### Definitions

The term “mammal” is used in its usual biological sense. Thus, it specifically includes humans and non-human mammals such as dogs, cats, horses, donkeys, mules, cows, domestic buffaloes, camels, llamas, alpacas, bison, yaks, goats, sheep, pigs, elk, deer, domestic antelopes, and non-human primates as well as many other species.

“Subject” as used herein, means a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

“Subject suspected of having” means a subject exhibiting one or more clinical indicators of a disease or condition. In certain embodiments, the disease or condition is one or more fibroses, fibrotic conditions, or fibrotic symptoms. In certain embodiments, the disease or condition is scleroderma. In certain embodiments, the disease or condition is non-alcoholic steatohepatitis (NASH). In certain embodiments, the disease or condition is cirrhosis. In certain embodiments, the disease or condition is non-alcoholic fatty liver disease (NAFLD). In certain embodiments, the disease or condition is idiopathic pulmonary fibrosis. In certain embodiments, the disease or condition is atherosclerosis. In certain embodiments, the disease or condition is hepatitis, alcoholic fatty liver disease, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, scleroderma, pulmonary fibrosis, diffuse parenchymal lung disease, idiopathic interstitial fibrosis, diffuse interstitial fibrosis; interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components.

As used herein, “fibrosis” refers to the abnormal deposition of extracellular matrix proteins. Such proteins include but are not limited to collagen, elastin, fibronectin, laminin, keratin, keratin, keratin sulfate, fibrin, perlecan, agrin, or agrecan. As used herein, “collagen” refers to any one of the subtypes of collagen, including but not limited to Type I, II, III, IV, V, VI, VII, VIII, IX, X, XI, XII, XIII, XIV, XV, XVI, XVII, or XVIII. Exemplary collagen types and subtypes especially include Type I, Type Ia, Type II, Type III, Type IV, and Type V. As used herein, fibrosis may occur by itself or as a symptom or sequela of another condition. As used herein, fibrosis may result from a genetic condition, a genetic predisposition, an environmental insult, an injury, healing of an injury, an autoimmune condition, or a chronic inflammation, a chronic inflammatory condition, or another condition leading to abnormal or excessive deposition of extracellular matrix components. Fibrosis as referred to herein may be assessed by assaying for, or determining the presence or level of, one or more biomarkers. Biomarkers for the presence of fibrosis include, but are not limited to, expression of the Col1a1, Col3a1, ACTA2, ENPP2, and/or LGALS1 genes or any combination or product thereof. Diagnosis or assessment of fibrosis may further be made by determination of the presence or level of type I collagen and/or hydroxyproline or any combination or product thereof. Diagnosis or assessment of fibrosis may also be made by histological, histochemical, or immunohistochemical analysis of one or more samples from a subject.

“Glycogen storage disease” means any one or more of a group of disorders marked by dysfunction in the synthesis, transport, or utilization of glycogen, generally due to the loss of a necessary enzyme activity. Glycogen storage diseases are generally classified by type according to their symptoms and etiologies. Known types include GSD type 0 (aglycogenesis, glycogen synthase deficiency); GSD type 1 (von Gierke disease, glucose-6-phosphatase translocase/transporter deficiency, GSD I); GSD type 2 (Pompe disease, alpha-1-4-glucosidase deficiency, GSD II); GSD type 3 (Cori disease, Forbes disease, limit dextrinosis, debranching enzyme disease; amylo-1-6-glucosidase deficiency due to loss of glucosidase, and/or transferase activity, GSD III); GSD type 4 (Andersen disease, glycogen phosphorylase deficiency, brancher deficiency, amylopectinosis, glycogen branching enzyme deficiency; amylo-1,4 to 1,6 transglucosidase deficiency, GSD IV); GSD type 5 (McArdle disease; glycogen phosphorylase (muscle type) deficiency, GSD V); GSD type 6 (Hers disease; glycogen phosphorylase E (liver type) deficiency, GSD VI); GSD type 7 (Tarui disease; phosphofructokinase deficiency, GSD VII); GSD type 8, 9 (GSD with phosphorylase activation system defects; phosphorylase kinase (liver or muscle isoforms) deficiency, GSD VIII and GSD IX); GSD type 10 (cyclic AMP-dependent kinase deficiency, GSD X); GSD type 11 (Fanconi-Bickel syndrome; glucose transporter type 2 (GLUT2) deficiency, GSD XI); and GSD type 12 (aldolase A deficiency, GSD XII). Subtypes of glycogen storage diseases are also known, in particular GSD 1a, which results from mutations in the gene for glucose-6-phosphatase (G6PC) and leads to, among other symptoms, the excess accumulation of glycogen and lipids in liver tissue, hepatomegaly, hepatic adenomas, and hepatocellular carcinoma. Symptoms of glycogen storage diseases may include elevated or reduced blood sugar, insulin insensitivity, myopathies, as well as hepatic symptoms such as steatosis, hyperlipidemia, hypercholesterolemia, cardiomegaly, hepatomegaly, fibrosis, cirrhosis, hepatocellular adenoma, and hepatocellular carcinoma. Symptoms may also include insulin insensitivity, elevated or reduced blood glucose, renal dysfunction, and/or fibrosis.

As used herein, “inflammatory disease” refers to a disease or disorder that is characterized by inflammation. Exemplary inflammatory diseases include, but are not limited to, acne, acid reflux/heartburn, age related macular degeneration (AMD), allergy, allergic rhinitis, Alzheimer's disease, amyotrophic lateral sclerosis, anemia, appendicitis, arteritis, arthritis, asthma. atherosclerosis, autoimmune disorders, balanitis, blepharitis, bronchiolitis, bronchitis, a bullous pemphigoid, burn, bursitis, cancer, cardiac arrest, carditis, celiac disease, cellulitis, cervicitis, cholangitis, cholecystitis, chorioamnionitis, chronic obstructive pulmonary disease (COPD), cirrhosis, colitis, congestive heart failure, conjunctivitis, cyclophosphamide-induced cystitis, cystic fibrosis, cystitis, common cold, dacryoadenitis, dementia, dermatitis, dermatomyositis, diabetes, diabetic neuropathy, diabetic retinopathy, diabetic nephropathy, diabetic ulcer, digestive system disease, eczema, emphysema, encephalitis, endocarditis, endometritis, enteritis, enterocolitis, epicondylitis, epididymitis, fasciitis, fibromyalgia, fibrosis, fibrositis, gastritis, gastroenteritis, gingivitis, glomerulonephritis, glossitis, heart disease, heart valve dysfunction, hepatitis, hidradenitis suppurativa, Huntington's disease, hyperlipidemic pancreatitis, hypertension, ileitis, infection, inflammatory bowel disease, inflammatory cardiomegaly, inflammatory neuropathy, insulin resistance, interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), a migraine, multiple sclerosis, myelitis, myocarditis, myositis, nephritis, non-alcoholic steatohepatitis, obesity, omphalitis, oophoritis, orchitis, osteochondritis, osteopenia, osteomyelitis, osteoporosis, osteitis, otitis, pancreatitis, Parkinson's disease, parotitis, pelvic inflammatory disease, pemphigus vularis, pericarditis, peritonitis, pharyngitis, phlebitis, pleuritis, pneumonitis, polycystic nephritis, proctitis, prostatitis, psoriasis, pulpitis, pyelonephritis, pylephlebitis, renal failure, reperfusion injury, retinitis, rheumatic fever, rhinitis, salpingitis, sarcoidosis, sialadenitis, sinusitis, spastic colon, stenosis, stomatitis, stroke, surgical complication, synovitis, tendonitis, tendinosis, tenosynovitis, thrombophlebitis, tonsillitis, trauma, traumatic brain injury, transplant rejection, trigonitis, tuberculosis, tumor, urethritis, ursitis, uveitis, vaginitis, vasculitis, and vulvitis. Inflammation as referred to herein may be assessed by assaying for, or determining the presence or level of, one or more biomarkers. Biomarkers for the presence of inflammation include, but are not limited to, expression of the TNF, CARD15, IL4R, IL23R, CTLA4, ANXA1, ANXA2, LGALS3, and/or PTPN22 genes or any combination or product thereof.

The term “atherosclerosis” refers to a condition characterized by irregularly distributed lipid deposits in the intima of large and medium-sized arteries wherein such deposits provoke fibrosis and calcification. Atherosclerosis raises the risk of angina, stroke, heart attack, or other cardiac or cardiovascular conditions.

“Subject in need thereof” means a subject identified as in need of a therapy or treatment.

A therapeutic effect relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

The term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of phenol and/or phosphonate groups or groups similar thereto. One of ordinary skill in the art will be aware that the protonation state of any or all of these compounds may vary with pH and ionic character of the surrounding solution, and thus the present disclosure contemplates multiple charge states of each compound. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

### Pharmaceutical Compositions

The compounds useful as described above can be formulated into pharmaceutical compositions for use in treatment of the conditions described herein. In some embodiments, pharmaceutical compositions comprising Compound 1 may be formulated for oral administration. In some specific embodiments, pharmaceutical compositions comprising Compound 1 may be formulated as a tablet. In other specific embodiments, pharmaceutical compositions comprising Compound 1 may be formulated as a gelcap. Some embodiments of the pharmaceutical compositions described herein comprise: (a) a safe and therapeutically effective amount of Compound 1, or pharmaceutically acceptable salts thereof; and (b) a pharmaceutically acceptable carrier, diluent, excipient or combination thereof.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy.

The actual unit dose of Compound 1 described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, or from about 20 mg to about 300 mg, or from about 200 mg to about 2000 mg. In some embodiments, the actual unit dose is 0.1 mg. In some embodiments, the actual unit dose is 0.5 mg. In some embodiments, the actual unit dose is 1 mg. In some embodiments, the actual unit dose is 1.5 mg. In some embodiments, the actual unit dose is 2 mg. In some embodiments, the actual unit dose is 2.5 mg. In some embodiments, the actual unit dose is 3 mg. In some embodiments, the actual unit dose is 3.5 mg. In some embodiments, the actual unit dose is 4 mg. In some embodiments, the actual unit dose is 4.5 mg. In some embodiments, the actual unit dose is 5 mg. In some embodiments, the actual unit dose is 10 mg. In some embodiments, the actual unit dose is 20 mg. In some embodiments, the actual unit dose is 25 mg. In some embodiments, the actual unit dose is 250 mg or less. In some embodiments, the actual unit dose is 100 mg or less. In some embodiments, the actual unit dose is 70 mg or less.

In some embodiments, Compound 1 is administered at a dose in the range of about 1-50 mg/m² of the body surface area. In some embodiments, Compound 1 is administered at a dose in the range of about 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5-6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, 2.5-22.5, or 9.5-21.5 mg/m², of the body surface area. In some embodiments, Compound 1 is administered at a dose of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, Compound 1 is administered at a dose less than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, Compound 1 is administered at a dose greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the Compound 1 dose is about 0.1 mg-100 mg, 0.1 mg-50 mg, 0.1 mg-20 mg, 0.1 mg-10 mg, 0.5 mg-100 mg, 0.5 mg-50 mg, 0.5 mg-20 mg, 0.5 mg-10 mg, 1 mg-100 mg, 1 mg-50 mg, 1 mg-20 mg, 1 mg-10 mg, 2.5 mg-50 mg, 2.5 mg-20 mg, 2.5 mg-10 mg, or about 2.5 mg-5 mg. In some embodiments, the TR-β agonist compound dose is about 5 mg-300 mg, 5 mg-200 mg, 7.5 mg-200 mg, 10 mg-100 mg, 15 mg-100 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, the amount of Compound 1 administered is about 20 mg-60 mg, 27 mg-60 mg, 20 mg-45 mg, or 27 mg-45 mg. In some embodiments, the amount of Compound 1 administered is about 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the Compound 1 dose is greater than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the Compound 1 dose is about less than about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the Compound 1 dose is about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg.

### Soft-Gel Formulations

Compound 1 may be formulated as a liquid fill composition for oral administration to a subject in need thereof. In some embodiments, Compound 1 may be formulated into a soft-gel for oral administration to a subject in need thereof. The softgel for oral administration comprises the liquid fill and a capsule. In some embodiments, Compound 1 may be formulated into a liquid fill with one or more excipients.

In some embodiments, the liquid fill described herein may be used to fill hard gelatin or HPMC capsules. In some embodiments, the liquid fill described herein may be used to fillstarch-based polyvinyl alcohol thermoplastic capsules. In some embodiments, the liquid fill may be used to fill any suitable capsule known in the art.

In some embodiments, a particular excipient may comprise 1%, 2%, 3%, 4%, 5%, 10% 15%, 20%, 25%, 30%, 35%, 40%, 45%, 50%, 55%, 60%, 65%, 70%, 75%, 80%, 85% 90%, 91%, 92%, 93%, 94%, 95%, 96%, 97%, 98%, or 99% by weight of the liquid fill. In some embodiments, a particular excipient may comprise 5%-10% by weight of the liquid fill. In some embodiments, a particular excipient may comprise 10-20%, 20-30%, 30-40% 40-50%, 50-60%, 60-70%, 70-80%, 90-99%, 10-30%, 20%-40%, 30-50%, 40-60%, 50-70%, 60-80%, 70-90% or 80-99% by weight of the liquid fill. In some embodiments, the liquid fill comprises one excipient. In other embodiments, the liquid fill comprises two excipients. In still other embodiments, the liquid fill comprises three excipients. In yet other embodiments, the liquid fill comprises four or more excipients.

In some embodiments provided here, the excipients may be selected from Maisine® CC, Crodamol™ GTCC, Crodamol™ GMCC, Capmul PG-8, Labrasol® ALF, Capryol™ 90, Caproyl™ PGMC, Kolliphor® EL, Kolliphor® RH 40, Tween® 80, Miglyol 812N, Span 80, Span 20, Vitamin E TPGS, Gelucire 44/14, Oleic Acid, Phosphal 53 MCT, Capmul MCM, Labrafac Lipophyle WL 1349, polyethylene glycols such as PEG-400, and combinations thereof. The excipients are often known by their trade names, the descriptions of which are provided herein.

| Excipient Trade Name | |
|---|---|
| or Abbreviation | Description |
| Maisine ® CC | Glycerol/glyceryl monolinoleate; primarily |
| | of di-and triglycerides of linoleic and oleic |
| | acids |
| Crodamol ™ GTCC | Caprylic/capric triglyceride, mixed glyceryl |
| | esters primarily of caprylic and capric acids |
| Crodamol ™ GMCC | Mixture of monoglycerides and diglycerides |
| | of caprylic and capric acids |
| Capmul PG-8 | Propylene glycol monocaprylate; prepared by |
| | esterification of propylene glycol |
| Labrasol ® ALF | Caprylocapryoyl polyoxyl-8 glycerides; |
| | comprises mono-, di-and triglycerides and |
| | mono- and di- fatty acid esters of |
| | polyethylene glycol (PEG)-8 and free PEG-8, |
| | with caprylic (C8)-and capric acid (C10) |
| Capryol ™ 90 | Propylene glycol monocaprylate (type II), |
| | propylene glycol caprylate; consists of |
| | propylene glycol esters of caprylic acid (C8) |
| Caproyl ™ PGMC | Propylene glycol monocaprylate (type I), |
| | propylene glycol caprylate; consists of |
| | propylene glycol esters of caprylic acid (C8), |
| Kolliphor ® EL | Polyoxyl castor oil; mixture of polyethylene |
| | glycol ethers; prepared by reacting castor oil |
| | with ethylene oxide in a molar ratio of 1:35 |
| Kolliphor ® RH 40 | Polyoxyl castor oil; prepared by reacting |
| | castor oil with ethylene oxide in a molar ratio |
| | of 1:40 |
| Tween ® 80 | Polysorbate 80, Polyoxyethylenesorbitan |
| | monooleate |
| Miglyol 812N | Triglycerides, medium-chain; triglyceride |
| | ester of saturated coconut/palm-kernel oil |
| | derived caprylic and capric fatty acids and |
| | plant derived glycerol |
| Vitamin E TPGS | D-alphatocopheryl polyethylene glycol 1000 |
| | succinate; polyethylene glycol derivative of |
| | α-tocopherol |
| Span 80 | Sorbitan monooleate |
| Span 20 | Sorbitan monolaurate |
| Gelucire 44/14 | Lauroyl macrogol-32 glycerides; consists of |
| | a small fraction of mono, di-and |
| | triglycerides and mainly PEG-32 (MW 1500) |
| | mono-and diesters of lauric acid (C12) |
| Phosphal 53 MCT | 53% phosphatidylcholine in caprylic/capric |
| | triglycerides, alcohol, glyceryl stearate, oleic |
| | acid and ascorbyl palmitate |
| Capmul MCM | Glyceryl caprylate/caprate; Medium chain |
| | mono-and diglyceride of Cs and Cio chain |
| Labrafac Lipophyle | Triglycerides, medium-chain; medium-chain |
| WL 1349 | triglycerides of caprylic and capric acids |
| PEG | Polyethylene glycol, specified by the average |
| | number of monomeric ethylene glycol units |
| | (i.e., PEG-300, PEG-400, PEG-500) |

In some embodiments, the excipient may comprise Maisine®. In some specific embodiment, the excipient may be Maisine® CC. In other embodiments, the excipient may be a combination of Maisine® CC and one additional excipient. In yet other embodiments, the excipient may be a combination of Maisine® CC and two or more additional excipients. In some embodiments, the excipient may comprise Maisine® and Capmul PG-8. In other embodiments, the excipient may comprise Crodamol™ GTCC, Maisine® CC, and Caproyl™ PGMC. In yet other embodiments, the excipient may comprise Crodamol™ GTCC and Labrasol® ALF 60%. In some additional embodiments, the excipient may comprise Crodamol™ GTCC, Labrasol® ALF, and Maisine CC. In other embodiments, the excipient may comprise Maisine® CC and Caproyl™ PGMC. In yet other embodiments, the excipient may comprise Labrasol® ALF and Capryol PGMC. In some embodiments, the excipient may comprise Labrasol® and Koliphor® EL. In other embodiments, the excipient may comprise Labrasol® ALF, Koliphor® EL, and Tween® 80. In yet other embodiments, the excipient may comprise Koliphor® EL and Tween 80®. In some embodiments, the excipient may comprise Labrasol® ALF, Capryol™ PGMC, and PEG-400. In other embodiments, the excipient may comprise Labrasol.

In some embodiments, the excipient comprises 20% to 60% by weight of the liquid fill. In some embodiments, the excipient comprises 25% to 65% Maisine® CC by weight of the liquid fill. In some embodiments, the excipient comprises 20% to 70% Maisine® CC by weight of the liquid fill. In some embodiments, the excipient comprises 20% to 80% Maisine® CC by weight of the liquid fill. In some embodiments, the excipient comprises 10% to 90% Maisine® CC by weight of the liquid fill. In some embodiments, the excipient comprises 10% to 95% Maisine® CC by weight of the liquid fill. In some embodiments, the excipient comprises 5% to 100% Maisine® CC by weight of the liquid fill.

In some embodiments, Maisine® CC comprises about 30%, 35%, 40%, 45%, 50%, 55%, 60%, 65%, 70%, 75%, 80%, 85%, 90%, 91%, 92%, 93%, 94%, 95%, 96%, 97%, 98%, 99% or greater than 99% by weight of the liquid fill present in the softgel dosage form, or within a range defined by the aforementioned values. In some embodiments, Maisine® CC comprises from about 90% to about 100% of the liquid fill present in the softgel dosage form. In some embodiments, Maisine® CC comprises from about 90% to about 100% of the liquid fill present in the softgel dosage form. In some embodiments, Maisine® CC comprises from about 95% to about 100% of the liquid fill present in the softgel dosage form. In some embodiments, Maisine® CC comprises from about 96% to about 100% of the liquid fill present in the softgel dosage form. In some embodiments, Maisine® CC comprises from about 97% to about 100% of the liquid fill present in the softgel dosage form. In some embodiments, Maisine® CC comprises from about 98% to about 100% of the liquid fill present in the softgel dosage form. In some embodiments, Maisine® CC comprises from about 99% to about 100% of the liquid fill present in the softgel dosage form.

In some embodiments, Crodamol GMCC comprises about 5%, 10%, 15%, 20%, 25%, 30%, 35%, 40%, 45%, or 50% by weight of the liquid fill present in the softgel dosage form, or within a range defined by the aforementioned values. In some embodiments, Crodamol GMCC comprises from about 20% to about 50% of the liquid fill present in the softgel dosage form. In some embodiments, Crodamol GMCC comprises from about 25% to about 40% of the liquid fill present in the softgel dosage form.

In some embodiments, Caproyl PGMC comprises about 20%, 25%, 30%, 35%, 40%, 45%, 50%, 55%, 60%, 65%, or 70% by weight of the liquid fill present in the softgel dosage form, or within a range defined by the aforementioned values. In some embodiments, Caproyl PGMC comprises from about 20% to about 70% of the liquid fill present in the softgel dosage form. In some embodiments, Caproyl PGMC comprises from about 30% to about 60% of the liquid fill present in the softgel dosage form. In some embodiments, Caproyl PGMC comprises from about 30% to about 50% of the liquid fill present in the softgel dosage form.

In some embodiments, Labrasol comprises about 20%, 25%, 30%, 35%, 40%, 45%, 50%, 55%, 60%, 65%, or 70% by weight of the liquid fill present in the softgel dosage form, or within a range defined by the aforementioned values. In some embodiments, Labrasol comprises from about 20% to about 70% of the liquid fill present in the softgel dosage form. In some embodiments, Labrasol comprises from about 30% to about 60% of the liquid fill present in the softgel dosage form. In some embodiments, Labrasol comprises from about 30% to about 50% of the liquid fill present in the softgel dosage form.

In some embodiments, Kolliphor EL comprises about 20%, 25%, 30%, 35%, 40%, 45%, 50%, 55%, 60%, 65%, 70%, 75%, or 80% by weight of the liquid fill present in the softgel dosage form, or within a range defined by the aforementioned values. In some embodiments, Kolliphor EL comprises from about 20% to about 70% of the liquid fill present in the softgel dosage form. In some embodiments, Kolliphor EL comprises from about 30% to about 60% of the liquid fill present in the softgel dosage form. In some embodiments, Kolliphor EL comprises from about 30% to about 50% of the liquid fill present in the softgel dosage form.

In some embodiments, Tween 80 comprises about 20%, 25%, 30%, 35%, 40%, 45%, or 50% by weight of the liquid fill present in the softgel dosage form, or within a range defined by the aforementioned values. In some embodiments, Tween 80 comprises from about 20% to about 40% of the liquid fill present in the softgel dosage form. In some embodiments, Tween 80 comprises from about 25% to about 35% of the liquid fill present in the softgel dosage form.

In some embodiments, PEG 400 comprises at least about 5%, 10%, 15%, 20%, 25%, or 30% by weight of the liquid fill present in the softgel dosage form, or within a range defined by the aforementioned values. In some embodiments, PEG 400 comprises from about 5% to about 30% of the liquid fill present in the softgel dosage form. In some embodiments, PEG 400 comprises from about 10% to about 30% of the liquid fill present in the softgel dosage form. In some embodiments, PEG 400 comprises from about 10% to about 20% of the liquid fill present in the softgel dosage form.

In some embodiments, the liquid fill may include one excipient. In some specific embodiments, the excipient may be Maisine.

In some embodiments, the liquid fill may include two excipients. In some embodiments, the excipients may be Crodamol GMCC and Labrasol ALF. In certain embodiments, the Crodamol GMCC and Labrasol ALF may be present in a ratio of 40:60 by weight. In other embodiments, the excipients may be Labrasol ALF and Kolliphor EL. In certain embodiments, the Labrasol ALF and Kolliphor EL may be present in a ratio of 50:50 by weight. In certain other embodiments, the Labrasol ALF and Kolliphor EL may be present in a ratio of 30:70 by weight. In some embodiments, the excipients may be Maisine and Caproyl PGMC. In certain embodiments, the Maisine and Caproyl PGMC may be present in a ratio of 60:40 by weight. In certain other embodiments, the Maisine and Caproyl PGMC may be present in a ratio of 40:60 by weight.

In some embodiments, the liquid fill may include three excipients. In some embodiments, the excipients may be Crodamol GMCC, Maisine CC and Caproyl PGMC. In certain embodiments the Crodamol GMCC, Maisine CC and Caproyl PGMC in a ratio of 30:30:40 by weight. In certain embodiments the Crodamol GMCC, Maisine CC and Caproyl PGMC in a ratio of 20:40:40 by weight. In some embodiments, the excipients may be Crodamol GMCC, Labrasol ALF, and Maisine CC. In certain embodiments the Crodamol GMCC, Labrasol ALF, and Maisine CC in a ratio of 25:35:40 by weight. In certain embodiments the Crodamol GMCC, Labrasol ALF, and Maisine CC in a ratio of 30:40:30 by weight.

In some embodiments, Compound 1 may be present in the liquid fill in the softgel dosage form at about 1%, 1.5%, 2%, 2.5%, 3%, 3.5%, 4%, 4.5%, 5%, 5.5%, 6%, 6.5%, 7%, 7.5%, 8%, 8.5, 9%, 9.5%, 10%, 11%, 12%, 13%, 14%, 15%, 16%, 17%, 18%, 19%, 20%, 21%, 22%, 23%, 24%, or 25% by weight of the liquid fillor within a range defined by any two of the aforementioned amounts.

In some embodiments, Compound 1 may be present in the liquid fill in the softgel dosage form at about 0.001 mg/mg of liquid fill, 0.005 mg/mg of liquid fill, 0.01 mg/mg of liquid fill, 0.02 mg/mg of liquid fill, 0.03 mg/mg of liquid fill, 0.04 mg/mg of liquid fill, 0.05 mg/mg of liquid fill, 0.06 mg/mg of liquid fill, 0.07 mg/mg of liquid fill, 0.08 mg/mg of liquid fill, 0.09 mg/mg of liquid fill, 0.10 mg/mg of liquid fill 0.11 mg/mg of liquid fill 0.12 mg/mg of liquid fill 0.13 mg/mg of liquid fill, 0.14 mg/mg of liquid fill, or 0.15 mg/mg of liquid fill by weight of the liquid fill, or within a range defined by any two of the aforementioned amounts. For example, Compound 1 may be present in the liquid fill in an amount of 0.001 mg to 0.01 mg per mg of liquid fill, 0.01 mg to 0.10 mg per mg of liquid fill, 0.01 mg to 0.05 mg per mg of liquid fill, 0.02 mg to 0.05 mg per mg of liquid fill, 0.02 mg to 0.06 mg per mg of liquid fill, 0.01 mg to 0.03 mg per mg of liquid fill, 0.02 mg to 0.03 mg per mg of liquid fill, 0.04 mg to 0.06 mg per mg of liquid fill, 0.05 mg to 0.06 mg per mg of liquid fill.

In some embodiments, the softgel formulations described herein may further comprise an antioxidant. In some embodiments, the antioxidant may be vitamin E, ascorbic acid, sodium ascorbate, erythorbic acid, butylated hydroxyanisole (BHA), butylated hydroxytoluene (BHT), tert-butyl hydroquinone, cresol, p-cresol, or any combination thereof. In some specific embodiments, the antioxidant is butylated hydroxytoluene.

In some embodiments, the antioxidant may be present may be present in the liquid formulation present in the softgel dosage form at about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of the liquid formulation, or within a range defined by any two of the aforementioned amounts.

### Second Pharmaceutical Agents

The dosage forms of Compound 1 disclosed herein may be administered in combination with one or more second pharmaceutical agents. In some embodiments, the dosage forms of Compound 1 disclosed herein may be administered in combination with one second pharmaceutical agent. In some embodiments, the compounds described above may be administered in combination with two second pharmaceutical agents. In some embodiments, the compounds described above may be administered in combination with three or more second pharmaceutical agents.

In some embodiments, the dosage forms of Compound 1 presented herein may be administered simultaneously with one or more second pharmaceutical agents. In other embodiments, the dosage forms of Compound 1 of the present disclosure may be administered sequentially with one or more second pharmaceutical agents. In some embodiments, Compound 1 and the second pharmaceutical agent are included together in the dosage forms described herein.

In one aspect, the dosage forms of Compound 1 presented herein may be administered in combination with a peroxisome proliferator-activated receptor (PPAR) modulator. PPAR modulators are pharmaceutical compounds that may be used e.g., to lower triglyceride levels and blood sugar levels in a subject. PPAR modulators may be classified as PPARα modulators, PPARγ modulators, or PPARδ agonists. In some embodiments, the PPAR modulator may be

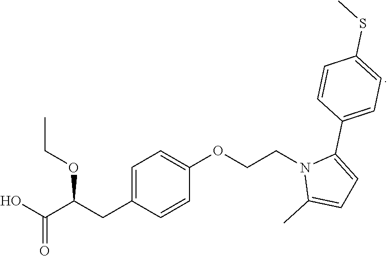 [SMILES: CCO[C@@H](Cc1ccc(OCCn2c(C)ccc2-c2ccc(SC)cc2)cc1)C(=O)O]

In some embodiments, the PPAR modulator may be

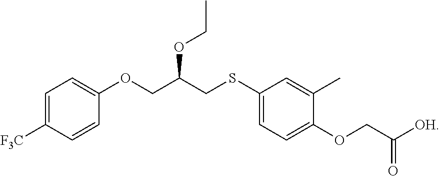 [SMILES: CCO[C@H](COc1ccc(C(F)(F)F)cc1)CSc1ccc(OCC(=O)O)c(C)c1]

In some embodiments, the PPAR modulator may be

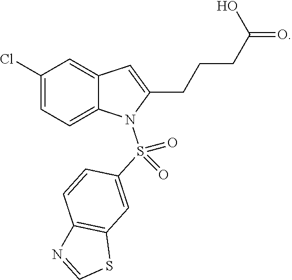 [SMILES: O=C(O)CCCc1cc2cc(Cl)ccc2n1S(=O)(=O)c1ccc2ncsc2c1]

In some embodiments, the PPAR modulator may be

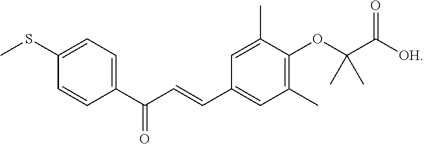 [SMILES: CSc1ccc(C(=O)/C=C/c2cc(C)c(OC(C)(C)C(=O)O)c(C)c2)cc1]

In some embodiments, the PPAR modulator may be a pharmaceutically acceptable salt or prodrug of any of the foregoing.

In some embodiments, the dosage forms of Compound 1 presented herein herein may be administered in combination with a fibric acid derivative. Fibric acid derivatives are a class of lipid-lowering drugs that have the ability to lower a subject's lipid profile. In some embodiments, the fibric acid derivative may be fenofibrate. In some embodiments, the fibric acid derivative may be gemfibrozil. In some embodiments, the fibric acid derivative may be fenofibric acid. In some embodiments, the fibric acid derivative may be clofibrate. In some embodiments, the fibric acid derivative may be a pharmaceutically acceptable salt or prodrug of any of the foregoing.

In some embodiments, the dosage forms of Compound 1 presented herein may be administered in combination with a bile acid receptor modulator. Bile acid receptors include, but are not limited to FXR (farnesoid X receptor) and TGR5. In some embodiments, the bile acid receptor modulator may be

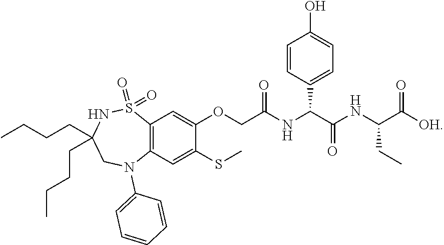 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

In some embodiments, the bile acid receptor modulator may be

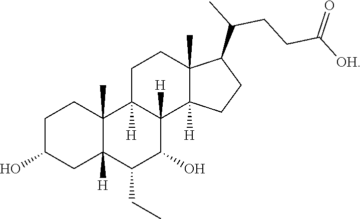

In some embodiments, the bile acid receptor modulator may be

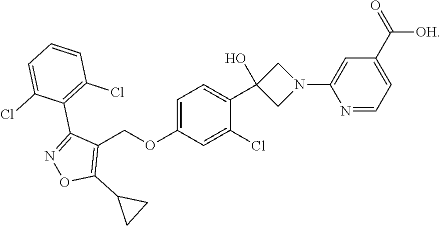 [SMILES: O=C(O)c1ccnc(N2CC(O)(c3ccc(OCc4c(-c5c(Cl)cccc5Cl)noc4C4CC4)cc3Cl)C2)c1]

In some embodiments, the bile acid receptor modulator may be

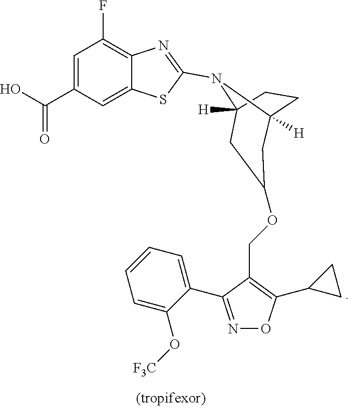 [SMILES: O=C(O)c1cc(F)c2nc(N3[C@@H]4CC[C@H]3CC(OCc3c(-c5ccccc5OC(F)(F)F)noc3C3CC3)C4)sc2c1]

In some embodiments, the bile acid modulator may be a pharmaceutically acceptable salt or prodrug of any of the foregoing.

In some embodiments, the dosage forms of Compound 1 presented herein may be administered in combination with a bile acid receptor modulator. In some embodiments, the bile acid receptor modulator may be selected from the group consisting of an FXR agonist, an FXR antagonist, a TGR agonist, and a dual FXR/TGR agonist. In some embodiments, the bile receptor acid modulator may be selected from a compound disclosed in Xu, J. Med. Chem. 2016, 59, 6553-6579, which is incorporated herein by reference in its entirety, including compounds selected from:

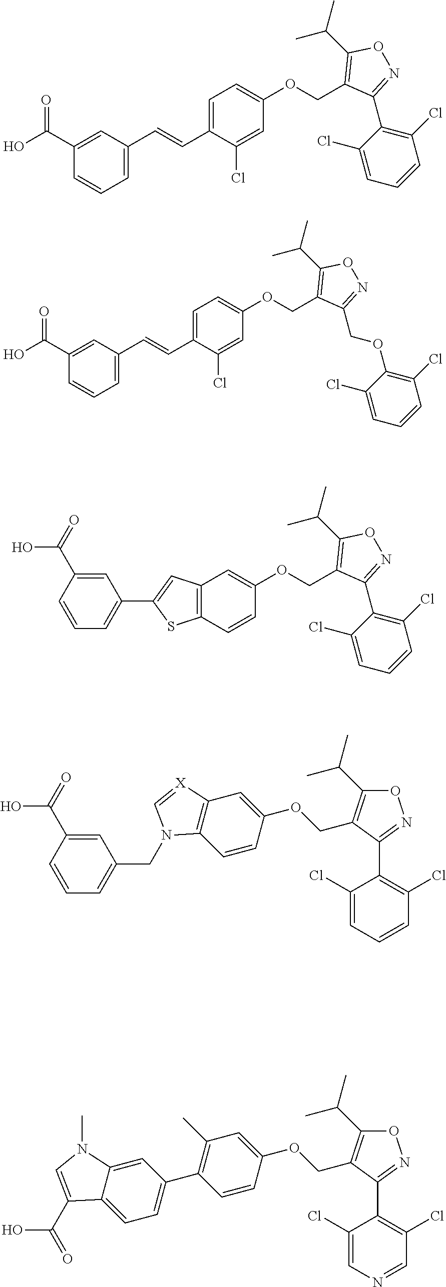

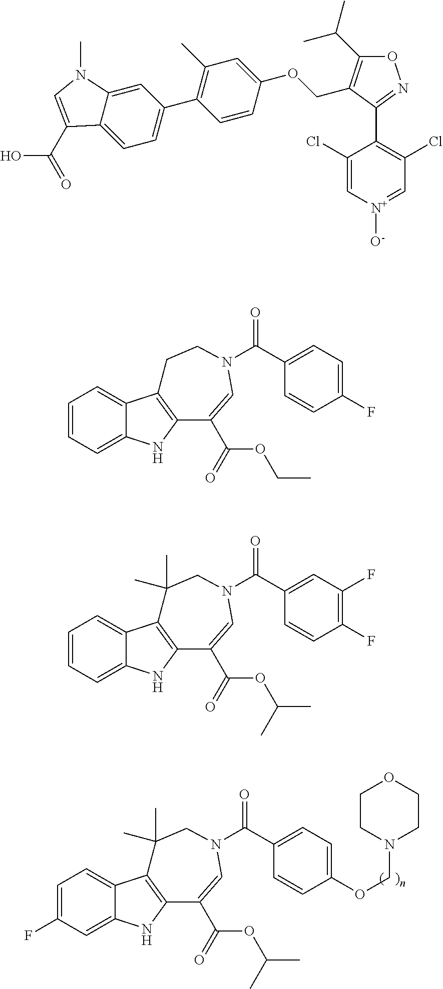

where n is 2 or 3

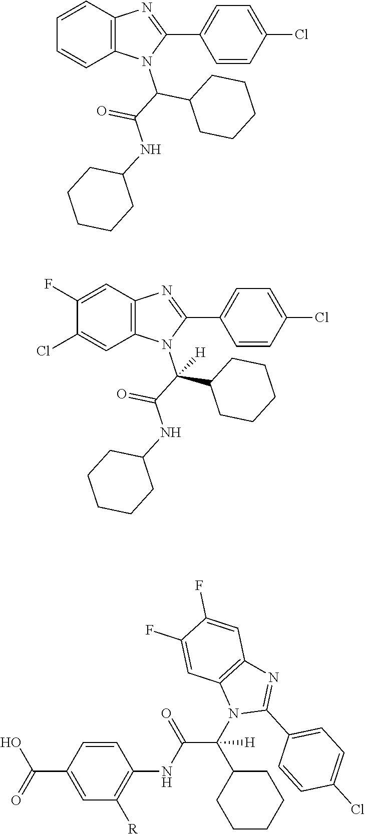

where R is H or F

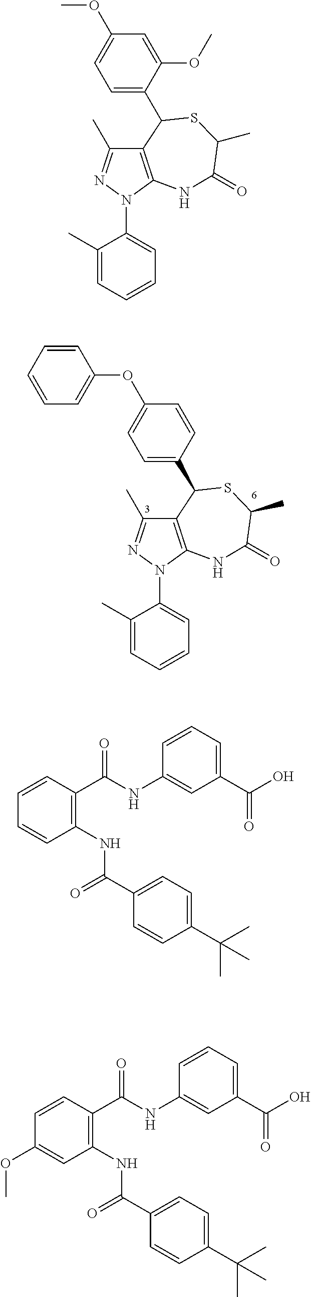

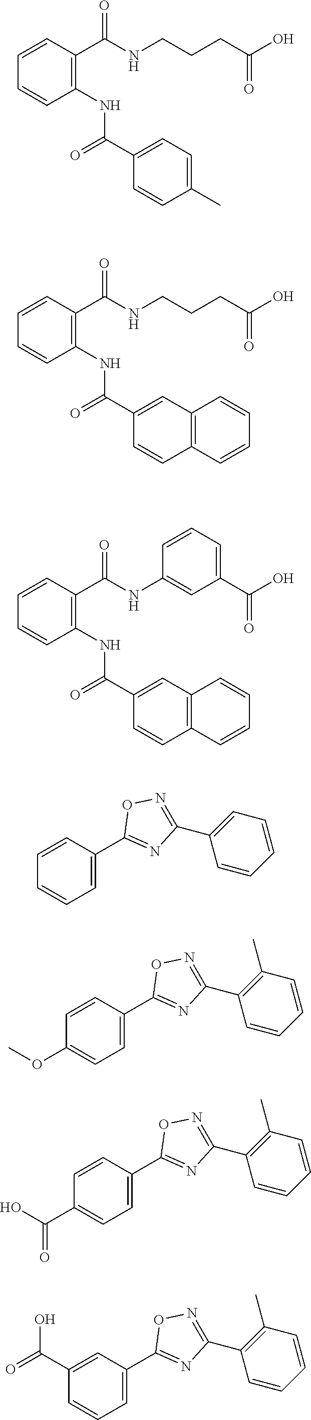

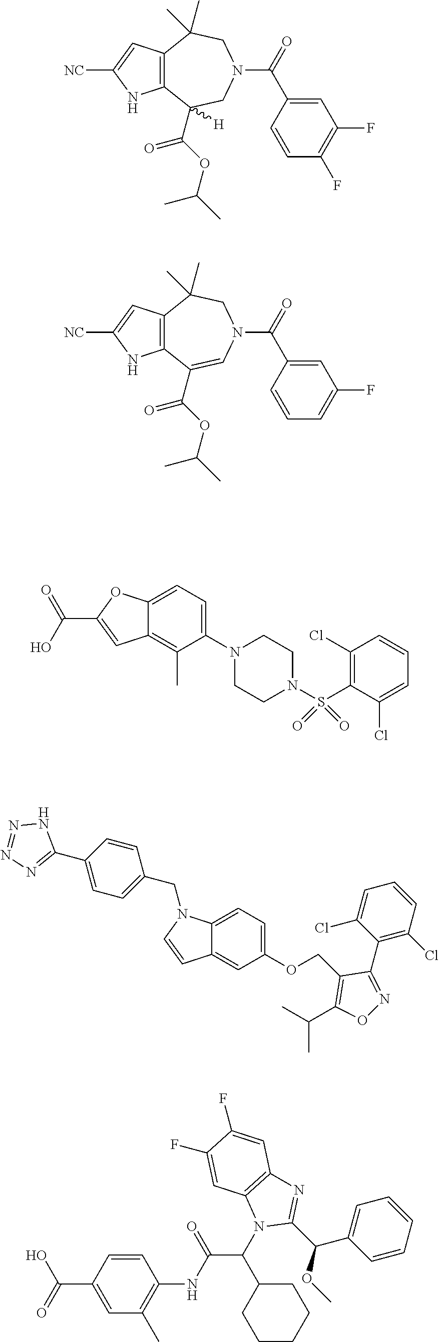

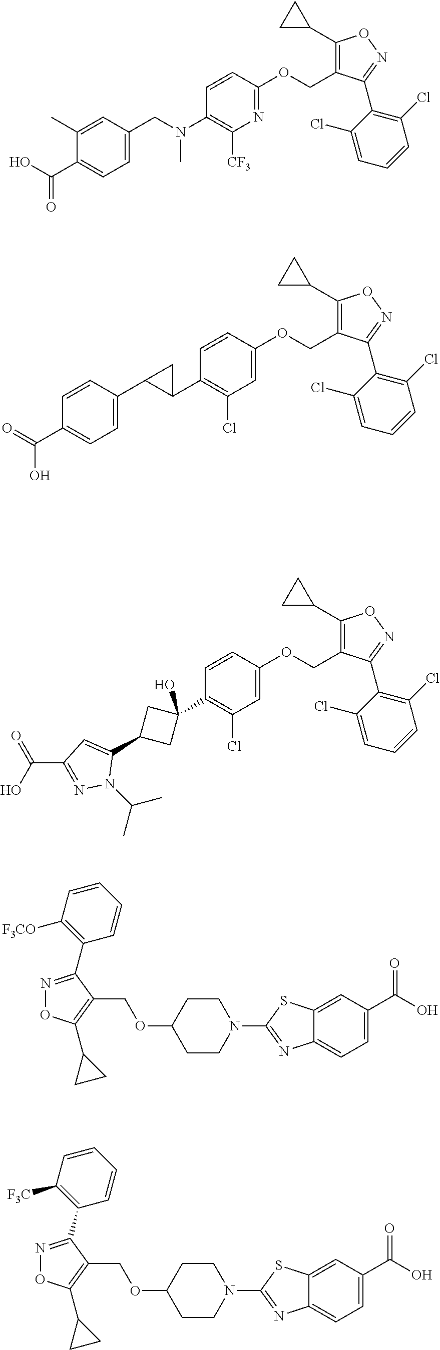

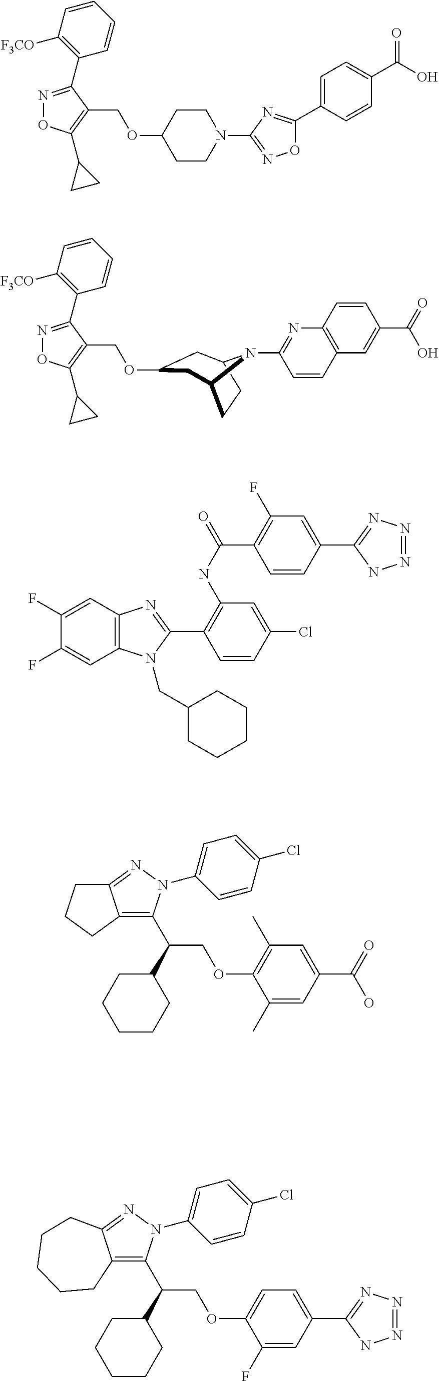

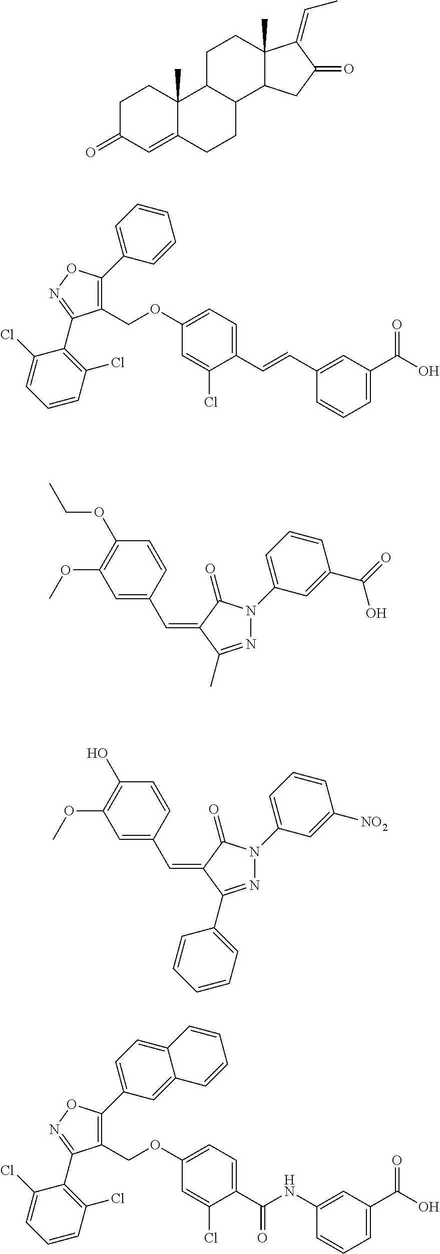

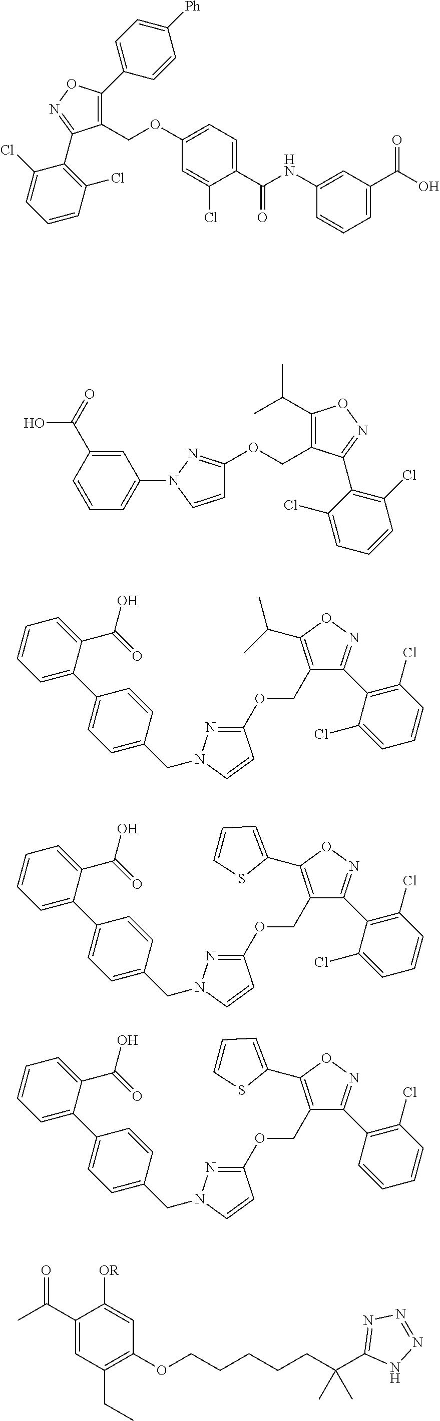

wherein R is H or benzyl

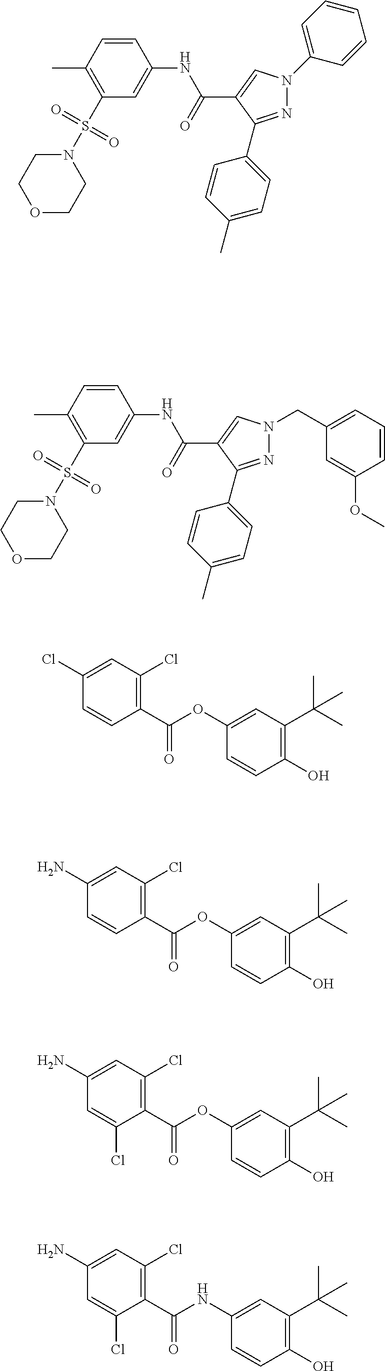

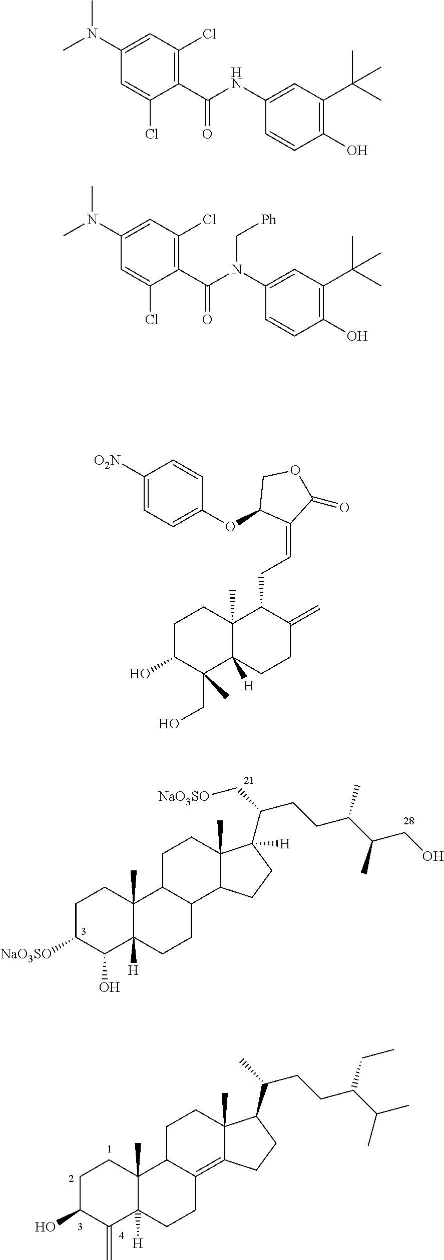

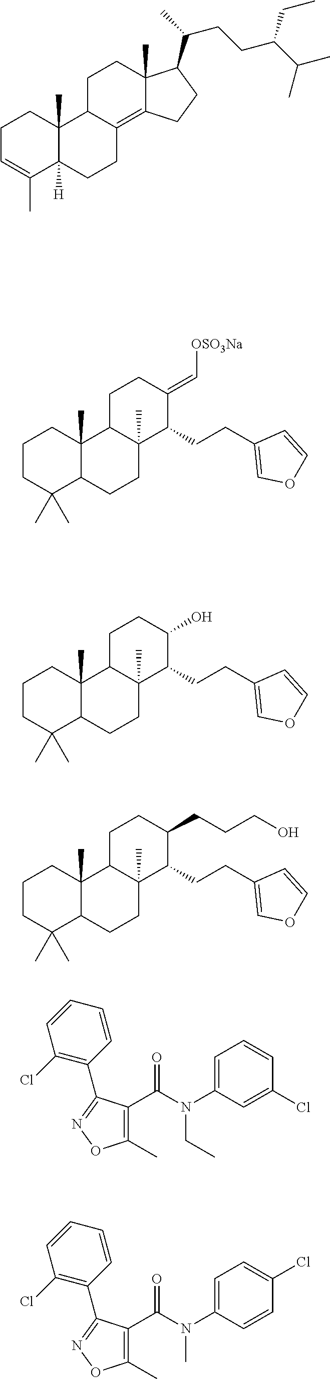

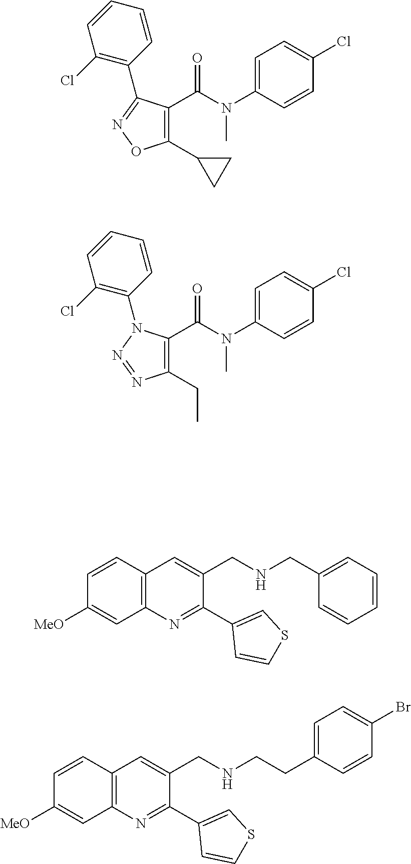

where R is H or methyl

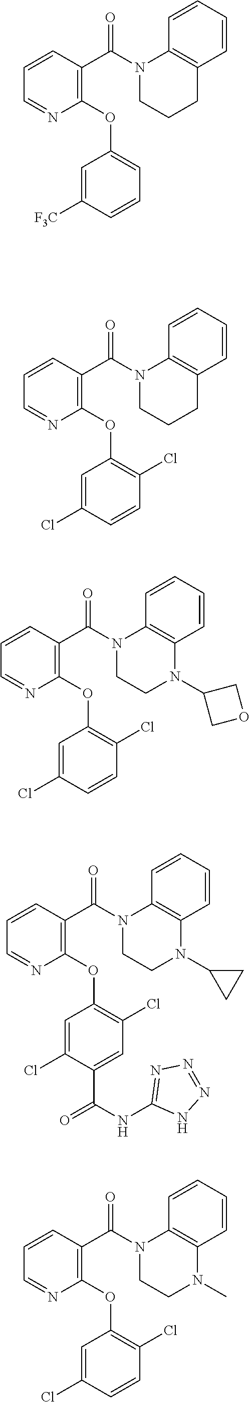

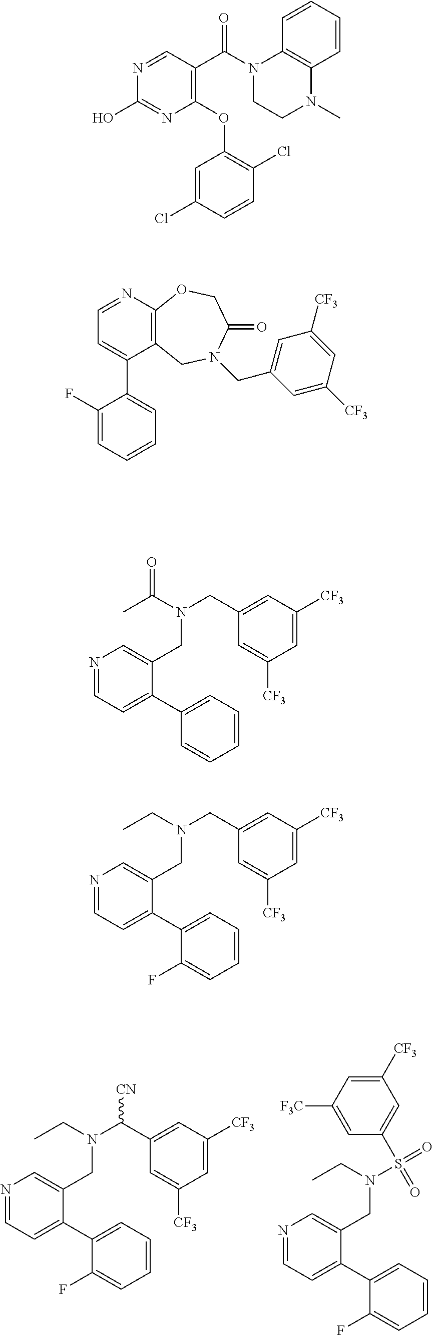

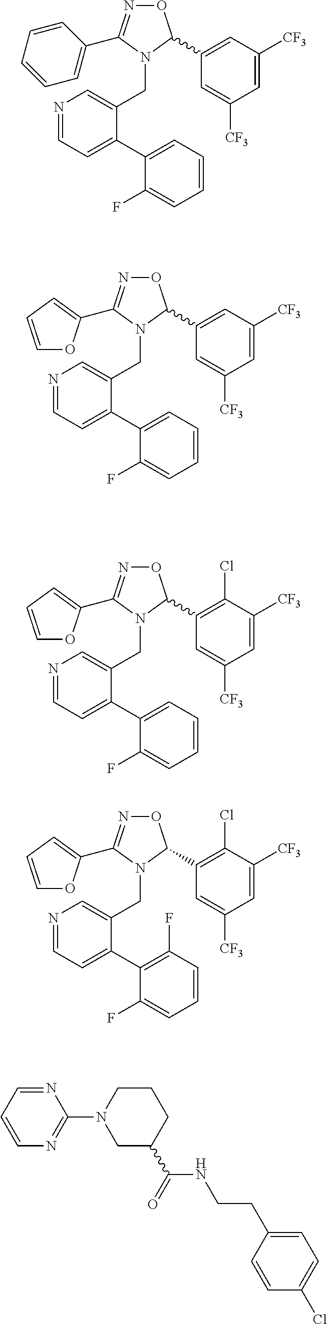

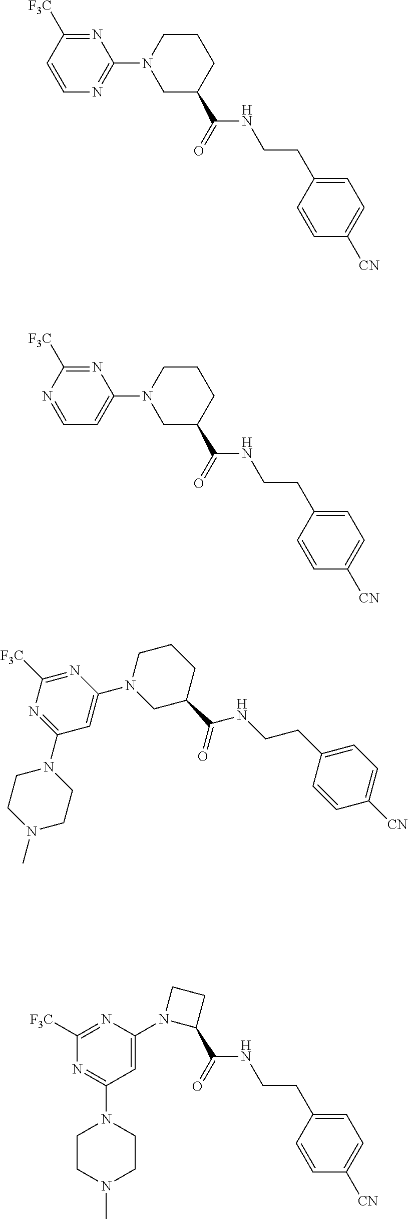

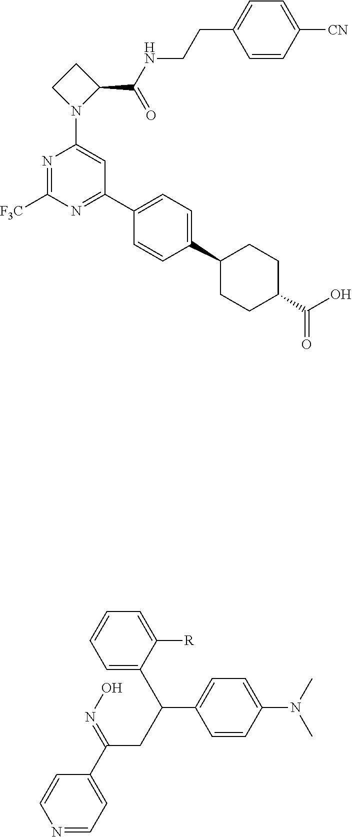

where R is H or methyl

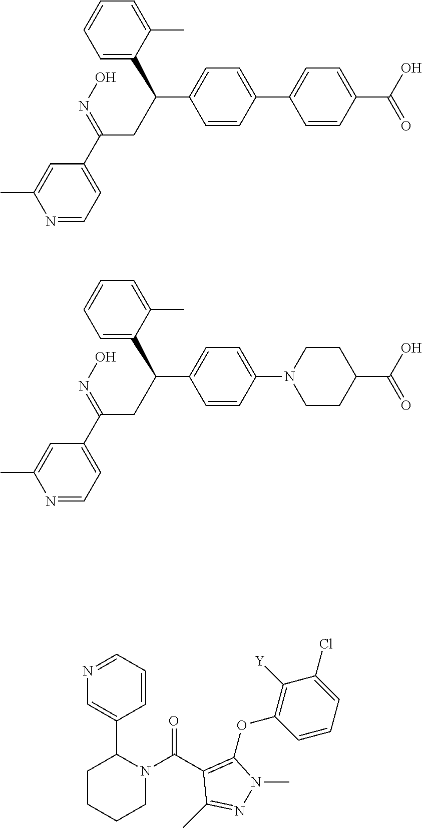

where Y is F or Cl

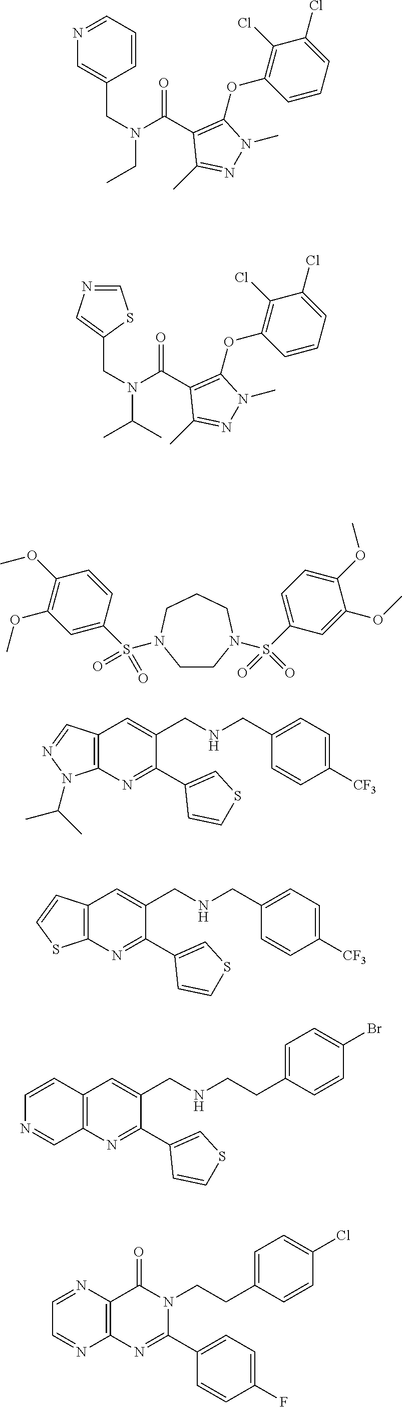

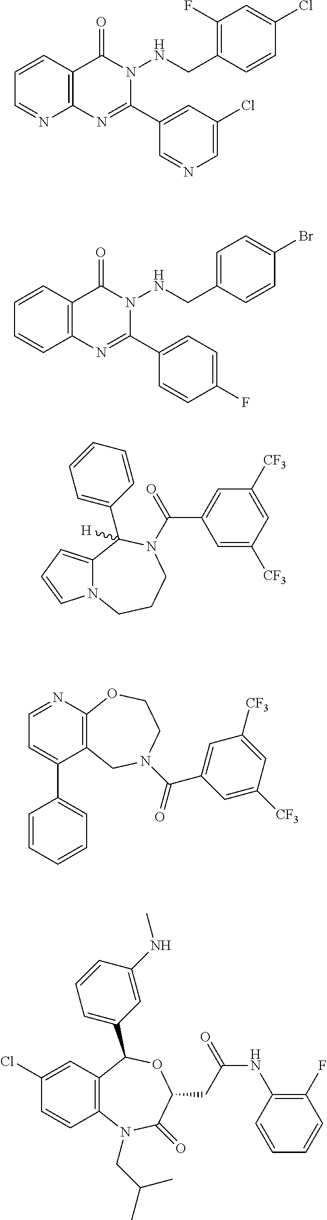

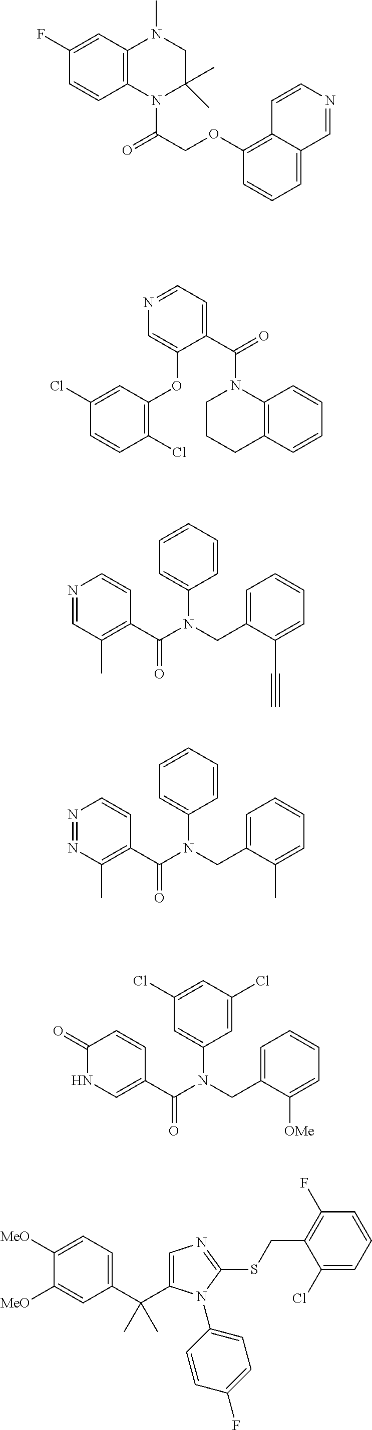

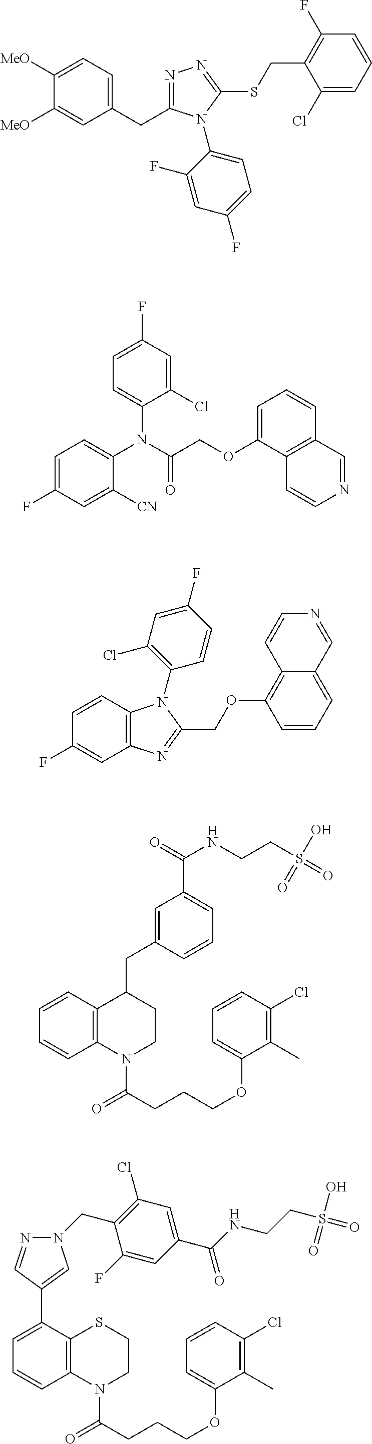

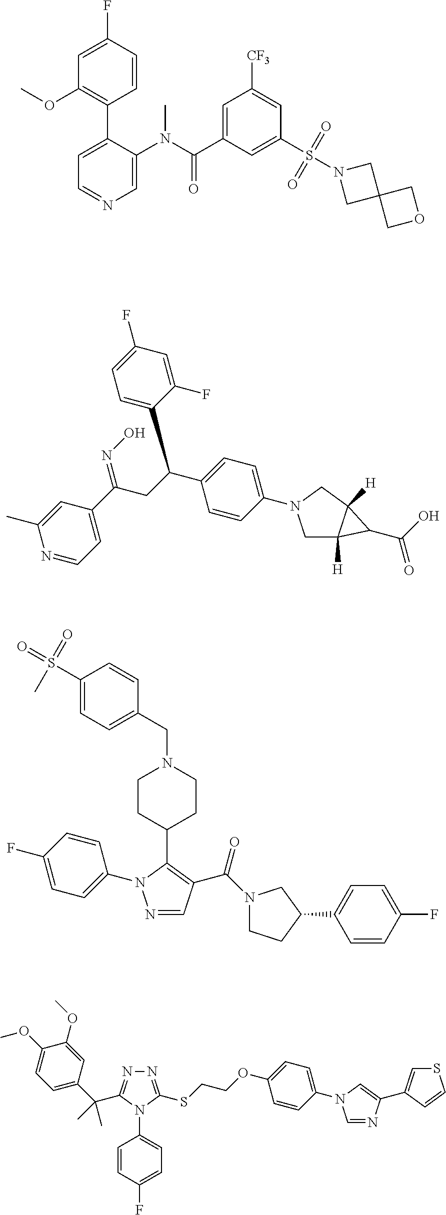

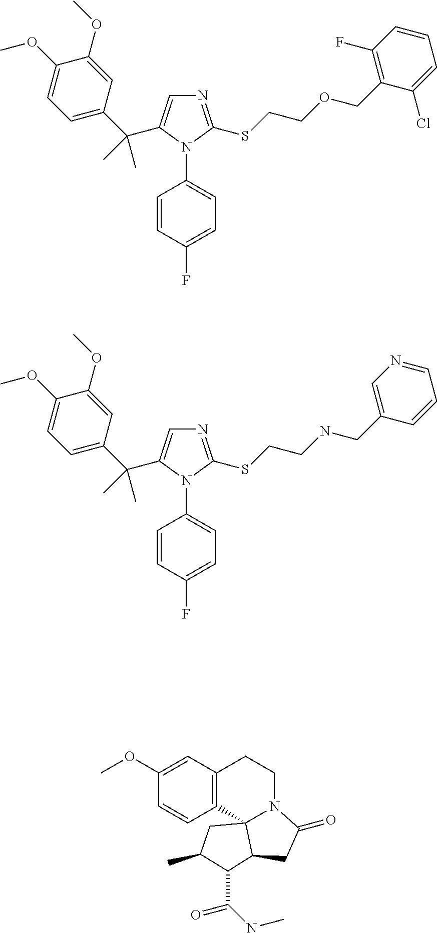

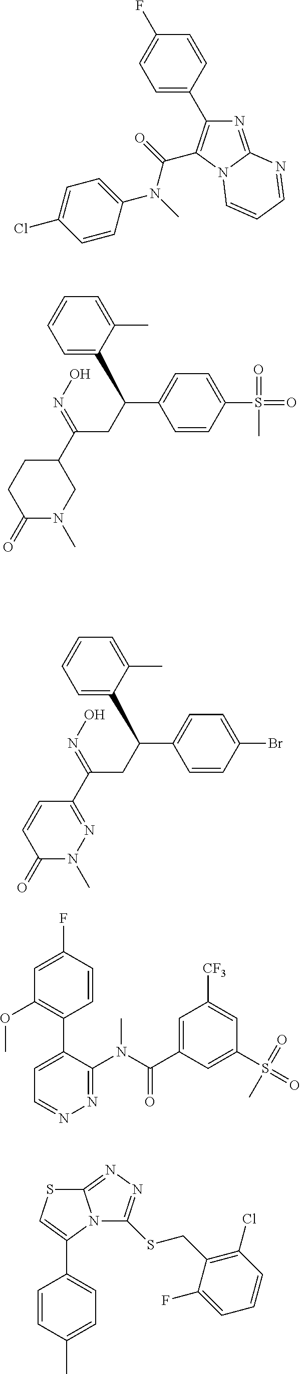

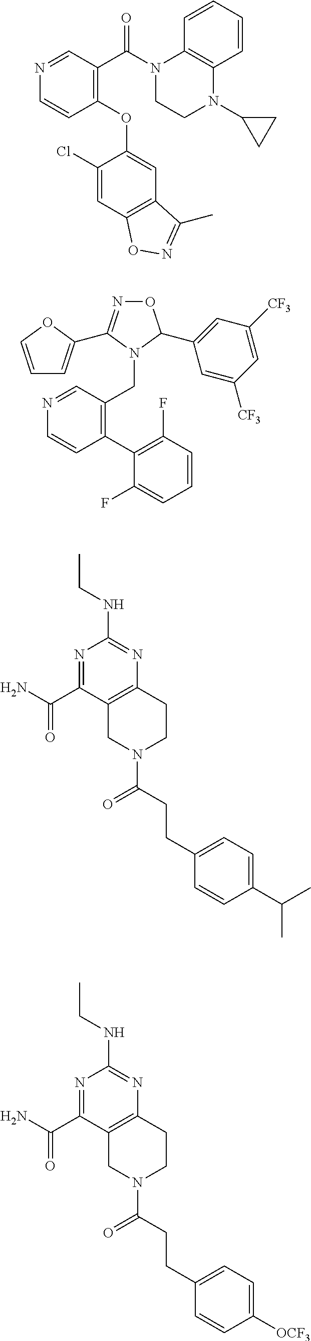

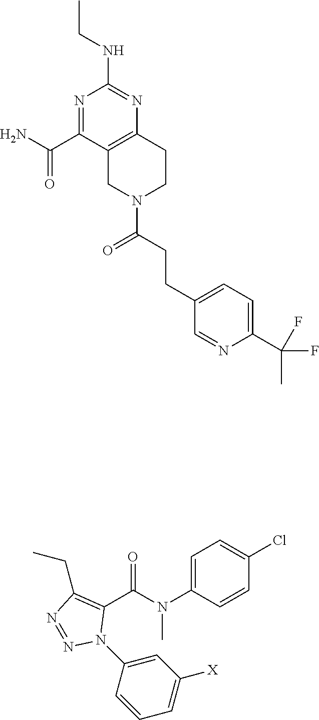

where X is H or OMe

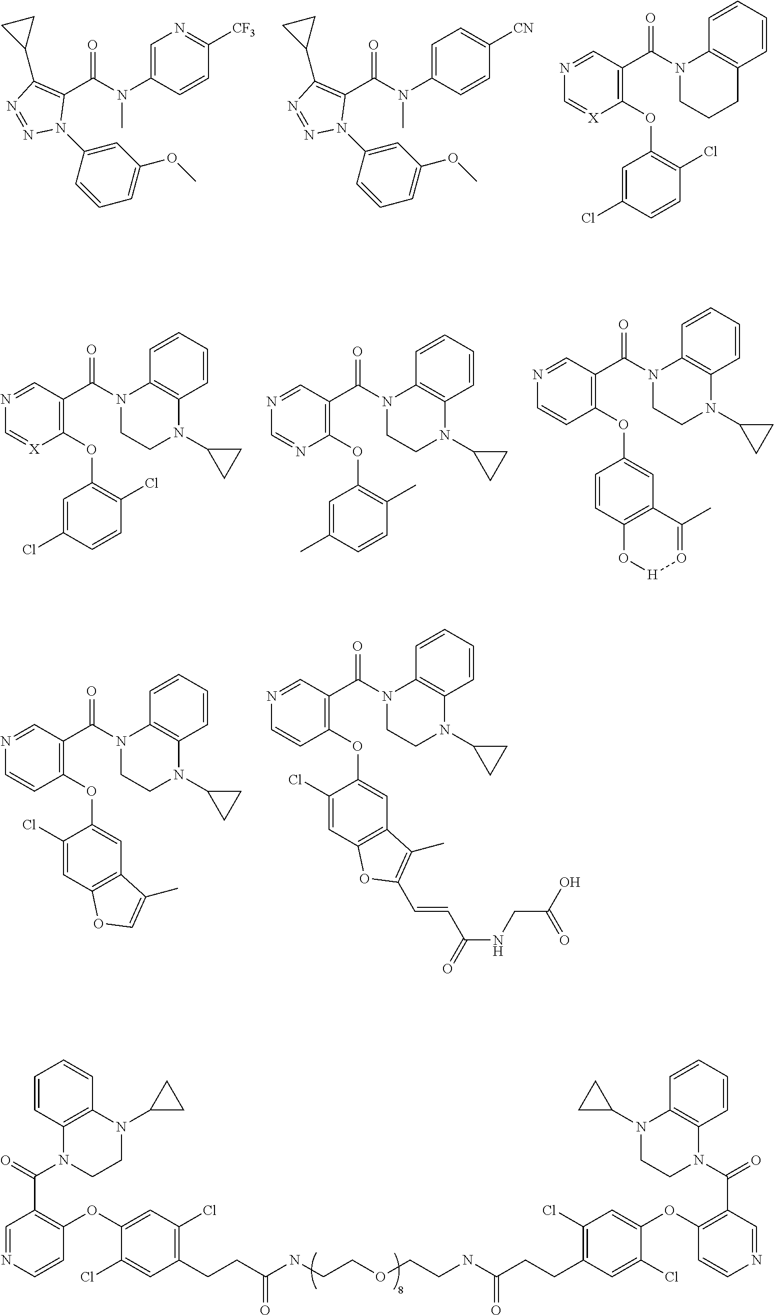

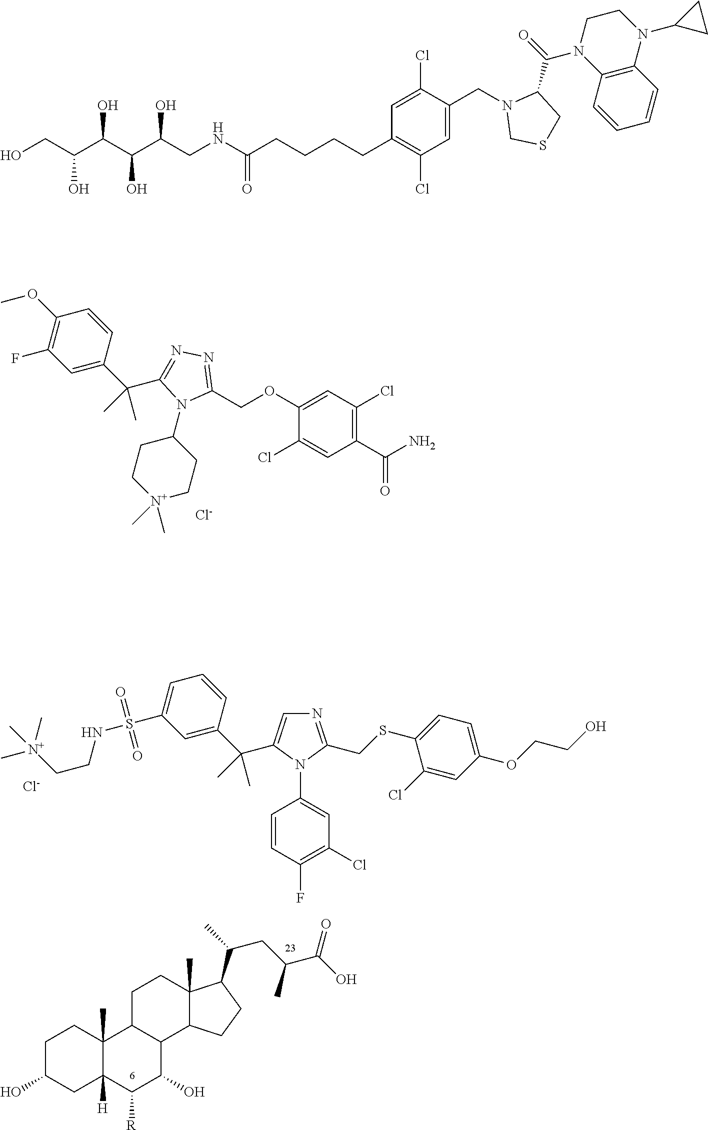

where R is H or ethyl

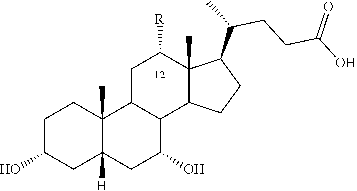

where R is H or OH

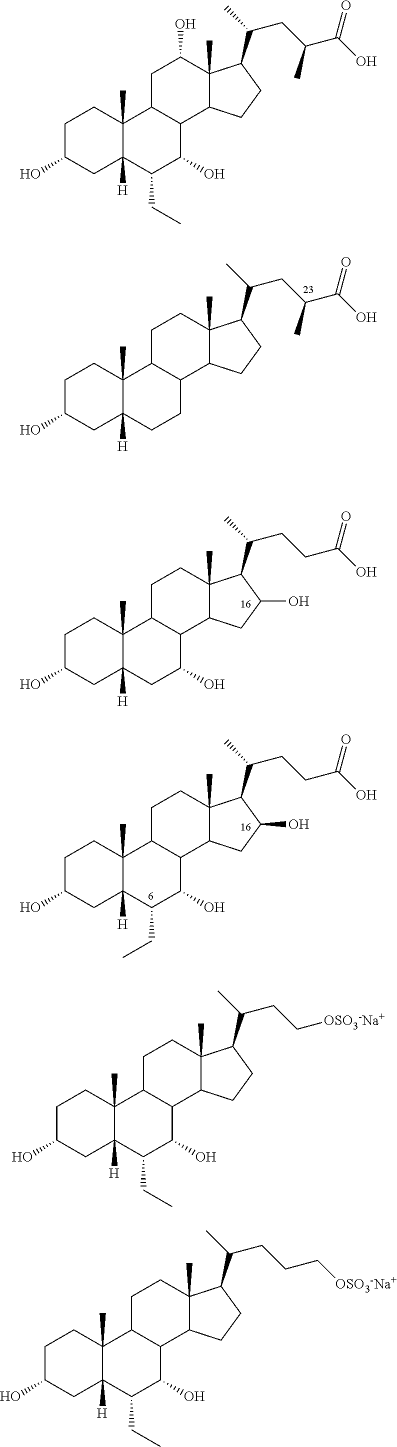

or pharmaceutically acceptable salts of any of the foregoing.

In some embodiments, the dosage forms of Compound 1 presented herein may be administered in combination with an anti-inflammatory compound. In some embodiments, the anti-inflammatory compound may be

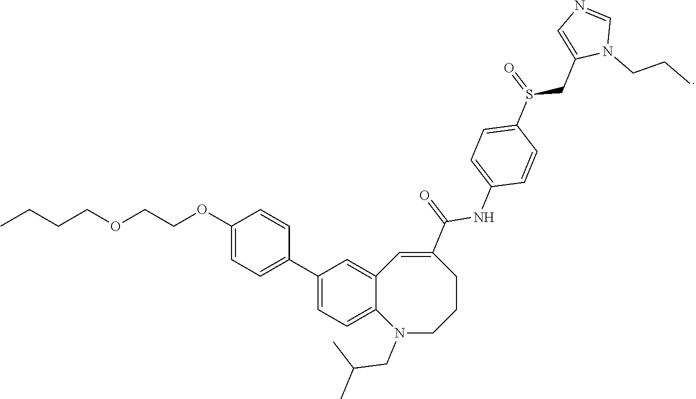 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

In some embodiments, the anti-inflammatory compound may be

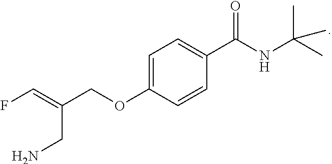 [SMILES: CC(C)(C)NC(=O)c1ccc(OC/C(=C/F)CN)cc1]

In some embodiments, the anti-inflammatory compound may be a poly-clonal or mono-clonal anti-LPS immunoglobulin. In some embodiments, the anti-LPS immunoglobulin may be IMM-124E. In some embodiments, the anti-inflammatory compound may be a pharmaceutically acceptable salt or prodrug of any of the foregoing.

In some embodiments, the second pharmaceutical agent may be an anti-fibrotic compound. In some embodiments, the anti-fibrotic compound may be

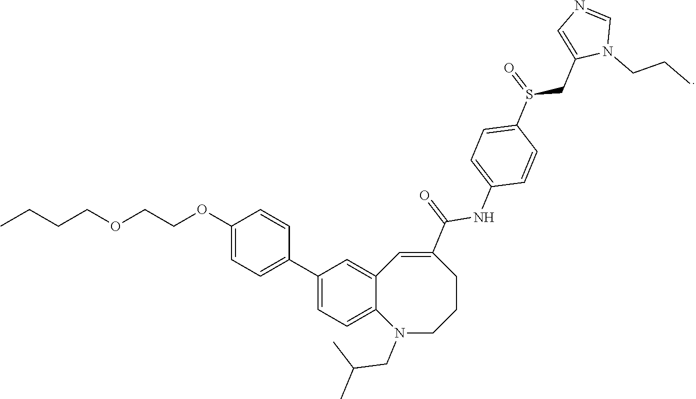 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

In some embodiments, the anti-fibrotic compound may be

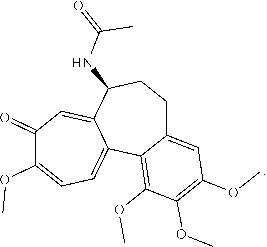

In some embodiments, the anti-fibrotic compound may be a pharmaceutically acceptable salt or prodrug of any of the foregoing.

In some embodiments, the dosage forms of Compound 1 presented herein may be administered in combination with a GLP-1 agonist. GLP-1 are pharmaceutical compounds that may be used e.g., to treat type 2 diabetes in a subject. In some embodiments, the GLP-1 agonist may be dulaglutide. In some embodiments, the GLP-1 agonist may be exenatide. In some embodiments, the GLP-1 agonist may be liraglutide. In some embodiments, the GLP-1 agonist may be albiglutide. In some embodiments, the GLP-1 agonist may be lixisenatide. In some embodiments, the GLP-1 agonist may be semaglutide. In some embodiments, the GLP-1 agonist may be insulin glargine. In some embodiments, the GLP-1 agonist is

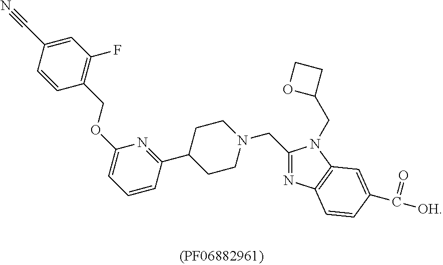 [SMILES: N#Cc1ccc(COc2cccc(C3CCN(Cc4nc5ccc(C(=O)O)cc5n4CC4CCO4)CC3)n2)c(F)c1]

In some embodiments, the GLP-1 agonist may be a pharmaceutically acceptable salt or prodrug of any of the foregoing.

In some embodiments, the dosage forms of Compound 1 presented herein may be administered in combination with a GLP-1 metabolic modulator. In some embodiments, the metabolic modulator may be a thyroid hormone receptor agonist. In other embodiments, the metabolic modulator may be a selective androgen receptor modulator. In some embodiments, the metabolic modulator may be a mitochondrial membrane transport protein modulator. In other embodiments, the metabolic modulator may be a selective estrogen receptor modulator. In some embodiments, the metabolic modulator may be an inhibitor of stearoyl-CoA desaturase 1 (SCD1). In some embodiments, the metabolic modulator may be an inhibitor of dipeptidyl peptidase 4 (DPP-4). In some embodiments, the metabolic modulator may be an inhibitor of sodium glucose cotransporters 1 and/or 2 (SGLT1, SGLT2, or dual SGLT1/SGLT2 inhibitors). In some embodiments, the metabolic modulator may be recombinant fibroblast growth factor 19 (FGF19) or engineered analogs, or recombinant fibroblast growth factor 21 (FGF21) or pegylated variants thereof. In some embodiments, metabolic the modulator may be

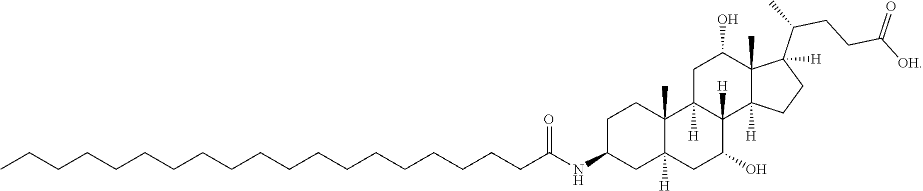

In some embodiments, the metabolic modulator may be

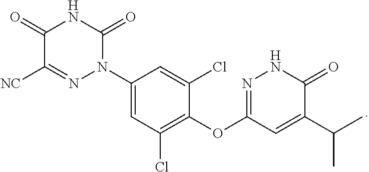 [SMILES: CC(C)c1cc(Oc2c(Cl)cc(-n3nc(C#N)c(=O)[nH]c3=O)cc2Cl)n[nH]c1=O]

In some embodiments, the metabolic modulator may be

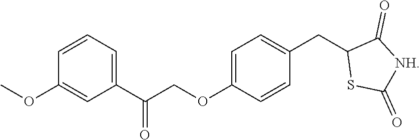 [SMILES: COc1cccc(C(=O)COc2ccc(CC3SC(=O)NC3=O)cc2)c1]

In some embodiments, the metabolic modulator may be a pharmaceutically acceptable salt or prodrug of any of the foregoing.

In some embodiments, the dosage forms of Compound 1 presented herein may be administered in combination with a fish oil derivative. Fish oils contain omega-3-fatty acids, which are polyunsaturated fatty acids (PUFAs) characterized by a double bond three atoms away from the terminal methyl group. They are widely distributed in nature and play an important role in the human diet and in human physiology, particularly with regard to lipid metabolism. In some embodiments, the fish oil derivative may be an omega-3-fatty acid alkyl ester. For example, the fish oil derivative may be an omega-3-fatty acid methyl ester, ethyl ester, n-propyl ester, or isopropyl ester. In some embodiments, the fish oil derivative may be an omega-3-fatty acid triglyceride. In some embodiments, the fish oil derivative may be ethyl (5Z,8Z,11Z,14Z,17Z)-eicosa-5,8,11,14,17-pentaenoate. In some embodiments, the fish oil derivative may be ethyl (4Z,7Z,10Z,13Z,16Z,19Z)-docosa-4,7,10,13,16,19-hexaenoate. In some embodiments, the fish oil derivative may be ethyl (7Z,10Z,13Z,16Z,19Z)-docosapentaenoate. In some embodiments, the fish oil derivative may be ethyl hexadecatrienoate. In some embodiments, the fish oil derivative may be α-linolenic acid ethyl ester. In some embodiments, the fish oil derivative may be ethyl (6Z,9Z,12Z,15Z)-6,9,12,15-octadecatetraenoate. In some embodiments, the fish oil derivative may be ethyl eicosatrienoate. In some embodiments, the fish oil derivative may be ethyl eicosatetraenoate. In some embodiments, the fish oil derivative may be ethyl heneicosapentaenoate. In some embodiments, the fish oil derivative may be ethyl icosapentaenoate. In some embodiments, the fish oil derivative may be ethyl heneicosapentaenoate. In some embodiments, the fish oil derivative may be ethyl tetracosapentaenoate. In some embodiments, the fish oil derivative may be nisinic acid ethyl ester. In some embodiments, the fish oil derivative may be a pharmaceutically acceptable salt or prodrug of any of the foregoing.

### Methods of Administration

In some embodiments, the composition may be administered one, twice, three times, our four times per day. In other embodiments, the composition may be administered once, twice, or three times per week. In other embodiments, the composition is administered every other day, every three days, or every four days. In other embodiments, the composition every other week, every three weeks, or every four weeks. In other embodiments, the composition is administered once per month or twice per month.

In some embodiments, an initial loading dose is administered which is higher than subsequent doses (maintenance doses). The dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. In some embodiments, the loading dose is 300 mg or less, 250 mg or less, 200 mg or less, 150 mg or less, 100 mg or less, 50 mg or less, 25 mg or less, 20 mg or less, 15 mg or less, 10 mg or less, or 5 mg or less. In some embodiments, the loading dose is 300 mg, 250 mg, 200 mg, 150 mg, 100 mg, 50 mg, 25 mg, 20 mg, 15 mg, 10 mg, 5, mg, or 2 mg. In some embodiments, the maintenance dose is 300 mg or less; 200 mg or less, 100 mg or less, 50 mg or less, 25 mg or less, 20 mg or less, 15 mg or less, 10 mg or less, 5 mg or less, 2.5 mg or less, or 1 mg or less. In some embodiments, the maintenance dose is 300 mg, 250 mg, 200 mg, 100 mg, 50 mg, 25 mg, 20 mg, 15 mg, 10 mg, 5 mg, 2.5 mg, or 1 mg.

### Methods of Treatment

Some embodiments according to the methods and compositions of the present disclosure relate to a method for preventing, treating, or ameliorating one or more fatty liver diseases in a subject comprising administering an effective amount of a Compound 1 described herein in combination with one or more second pharmaceutical agents to a subject in need thereof. In some embodiments, the fatty liver disease may be steatosis. In other embodiments, the fatty liver disease may be non-alcoholic fatty liver disease. In some embodiments, the fatty liver disease may be non-alcoholic steatohepatitis (NASH). In some embodiments, the subject may have two or more of the aforementioned fatty liver diseases.

Some embodiments according to the methods and compositions of the present disclosure relate to a method for the reduction or prevention of the deposition of extracellular matrix proteins, comprising administering an effective amount of Compound 1 described herein in combination with one or more second pharmaceutical agents described herein to a subject in need thereof. In some embodiments, said deposition of extracellular matrix proteins may comprise abnormal or excessive deposition of said proteins. In some embodiments, said extracellular matrix proteins may comprise one or more of collagen, keratin, elastin, or fibrin. In some embodiments, said extracellular matrix proteins may comprise collagen. In some embodiments, said extracellular matrix proteins may comprise Type I collagen. In some embodiments, said extracellular matrix proteins may comprise Collagen Type Ia. In some embodiments, said extracellular matrix proteins may comprise Type III collagen. Some embodiments according to the compositions and methods of the present disclosure relate to a method for the treatment of a fibrosis or its symptoms or sequelae, comprising administering an effective amount of a compound described herein to a subject in need thereof.

In some embodiments, the compounds and compositions comprising Compound 1 described herein and/or one or more second pharmaceutical agents described herein can be used to treat a variety of conditions arising from fibrosis or inflammation, and specifically including those associated with abnormal collagen deposition. Example conditions include glycogen storage disease type III (GSD III), glycogen storage disease type VI (GSD VI), glycogen storage disease type IX (GSD IX), non-alcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis, or symptoms or sequelae thereof, or other diseases or conditions resulting in the excessive deposition of extracellular matrix components, such as collagen.

In some embodiments the methods of the present disclosure comprise methods for the treatment, amelioration, or prevention of a fibrotic condition. In some embodiments, said fibrotic condition may be secondary to another condition. In some embodiments, said fibrotic condition or primary condition may further comprise chronic inflammation of an organ, tissue, spatial region, or fluid-connected area of the body of a subject. In some embodiments, said inflammation may comprise activation of one or more TGF-beta dependent signaling pathways. In some embodiments, said TGF-β dependent signaling pathways may comprise one or more elements responsive to T3 or T4. In some embodiments, said fibrotic condition may comprise abnormal or excessive deposition of one or more of collagen, keratin, or elastin. In some embodiments, said fibrotic condition may comprise abnormal or excessive deposition of collagen. In some embodiments, said fibrotic condition may comprise abnormal or excessive deposition of Type I collagen. In some embodiments, said fibrotic condition may comprise abnormal or excessive deposition of Collagen Type Ia. In some embodiments, said fibrotic condition may comprise abnormal or excessive deposition of Type III collagen. In some embodiments said fibrotic condition may comprise one or more of glycogen storage disease type III (GSD III), glycogen storage disease type VI (GSD VI), glycogen storage disease type IX (GSD IX), non-alcoholic steatohepatitis (NASH), cirrhosis, hepatitis, scleroderma, alcoholic fatty liver disease, atherosclerosis, asthma, cardiac fibrosis, organ transplant fibrosis, muscle fibrosis, pancreatic fibrosis, bone-marrow fibrosis, liver fibrosis, cirrhosis of liver and gallbladder, fibrosis of the spleen, scleroderma, pulmonary fibrosis, idiopathic pulmonary fibrosis, diffuse parenchymal lung disease, idiopathic interstitial fibrosis, diffuse interstitial fibrosis, interstitial pneumonitis, desquamative interstitial pneumonia, respiratory bronchiolitis, interstitial lung disease, chronic interstitial lung disease, acute interstitial pneumonitis, hypersensitivity pneumonitis, nonspecific interstitial pneumonia, cryptogenic organizing pneumonia, lymphocytic interstitial pneumonia, pneumoconiosis, silicosis, emphysema, interstitial fibrosis, sarcoidosis, mediastinal fibrosis, cardiac fibrosis, atrial fibrosis, endomyocardial fibrosis, renal fibrosis, chronic kidney disease, Type II diabetes, macular degeneration, keloid lesions, hypertrophic scar, nephrogenic systemic fibrosis, injection fibrosis, complications of surgery, fibrotic chronic allograft vasculopathy and/or chronic rejection in transplanted organs, fibrosis associated with ischemic reperfusion injury, post-vasectomy pain syndrome, fibrosis associated with rheumatoid arthritis, arthrofibrosis, Dupuytren's disease, dermatomyositis-polymyositis, mixed connective tissue disease, fibrous proliferative lesions of the oral cavity, fibrosing intestinal strictures, Crohn's disease, glial scarring, leptomeningeal fibrosis, meningitis, systemic lupus erythematosus, fibrosis due to radiation exposure, fibrosis due to mammary cystic rupture, myelofibrosis, retroperitoneal fibrosis, progressive massive fibrosis. In some embodiments, said fibrotic condition may comprise one or more of GSD III, GSD IX, Non Alcoholic Steatohepatitis, cirrhosis of the liver and/or pancreas, scleroderma, idiopathic pulmonary fibrosis, psoriasis, alcoholic fatty liver disease, Dupuytren's disease, and/or any combination thereof.

According to the methods and composition as disclosed herein, the fibrotic condition or condition having fibrosis as a sequela may further comprise chronic inflammation. According to the methods and compositions as disclosed herein, the fibrotic condition or condition having fibrosis as a sequela may further comprise activation of one or more TGF-β dependent signaling pathways. According to the methods and compositions as disclosed herein, the fibrotic condition or condition having fibrosis as a sequela may further comprise activation and/or repression of one or more Thyroid Receptor Beta (TRβ) dependent signaling pathways. According to the methods and compositions as disclosed herein, the fibrotic condition or condition having fibrosis as a sequela may further comprise the involvement of signaling pathways responsive to triiodothyronine (T3), thyroxine (T4), any combination thereof, or mimetics thereof. According to the methods and compositions as disclosed herein, the fibrotic condition or condition having fibrosis as a sequela may further comprise the involvement of receptors responsive to T3, T4, any combination thereof, or mimetics thereof. In some embodiments according to the methods and compositions disclosed herein, the fibrotic condition or condition having fibrosis as a sequela may comprise the involvement of TRβ.

In some embodiments, the compositions and methods described herein provide compositions and methods for the treatment, amelioration, prevention or cure of collagen deposition. In some embodiments, said collagen deposition comprises and abnormal or excessive deposition of collagen. In some embodiments, said collagen deposition may comprise abnormal or excessive deposition of Type I collagen. In some embodiments, said collagen deposition may comprise abnormal or excessive deposition of Collagen Type Ia. In some embodiments, said collagen deposition may comprise abnormal or excessive deposition of Type III collagen. According to the methods and compositions as disclosed herein, said collagen deposition may further comprise the involvement of receptors responsive to T3, T4, any combination thereof, or mimetics thereof. In some embodiments according to the methods and compositions disclosed herein, said collagen deposition may comprise the involvement of TRβ. In some embodiments according to the methods and compositions disclosed herein, said collagen deposition may be prevented, ameliorated, or cured by the administration of one or more agonists of TRβ.

In some embodiments, administration of dosage forms of Compound 1 described herein results in a reduction in the expression of the Cola1, Col3a1, αSMA, and/or Galectin1 genes or any combination or product thereof in the subject to which said combination is administered. In some embodiments, administration of dosage forms of Compound 1 described herein results in a reduction in the degree of fibrosis observable by histology, histochemistry, immunohistochemistry, or the like, and/or reductions in the amount, accumulation, or distribution of type 1 collagen and/or hydroxyproline or any combination thereof in the subject to which said combination is administered. In some embodiments, administration of dosage forms of Compound 1 described herein as disclosed herein results in a reduction in total serum lipids, total serum cholesterol, total serum triglycerides, total liver lipids, total liver cholesterol, total liver triglycerides, or any combination thereof.

### EXAMPLES

The compositions and methods described herein are further illustrated by the following non-limiting examples.

### Example 1: Preparation of Liquid Fill Compositions

A variety of liquid fill compositions of Compound 1 were prepared in order to determine the suitability of each system. The concentration of Compound 1 was 50 mg per gram of liquid fill The various compositions prepared are shown in Table 1 below.

TABLE 1
Initial Liquid Fill Formulations of Compound 1
Maisine (100%)

| Crodamol GMCC (30%) | Maisine CC (30%) | Capryol PGMC (40%) |
|---|---|---|
| Crodamol GMCC (20%) | Maisine CC (40%) | Capryol PGMC (40%) |
| Crodamol GMCC (40%) | Labrasol ALF (60%) | |
| Crodamol GMCC (25%) | Labrasol ALF (35%) | Maisine CC (40%) |
| Crodamol GMCC (30%) | Labrasol ALF (40%) | Maisine CC (30%) |
| Maisine CC (60%) | Capryol PGMC (40%) | |
| Maisine CC (40%) | Capryol PGMC (60%) | |
| Labrasol ALF (70%) | Capryol PGMC (30%) | |
| Labrasol (50%) | Koliphor EL (50%) | |
| Labrasol (30%) | Koliphor EL (70%) | |
| Labrasol (30%) | Kolliphor EL (30%) | Tween 80 (40%) |
| Kolliphor EL (70%) | Tween 80 (30%) | |
| Labrasol (50%) | Capryol PGMC (30%) | PEG400 (20%) |
| Labrasol (45%) | Kolliphor EL (45%) | PEG 400 (10%) |
| Labrasol (40%) | Kolliphor EL (40%) | PEG 400 (20%) |

### Example 2: Hydrolytic Stability Data for Compound 1 in Liquid Excipients

In order to evaluate the miscibility of excipients, first a placebo mix was prepared for each formulation. Once the miscibility of excipients was confirmed, 50 mg/g of Compound 1 formulation mixes were prepared and spiked with 5% w/w water in order to evaluate the effect that water migration during encapsulation would have on the hydrolytic impurities. The water spiked samples were stored at 40° C. Hydrolytic stability data is provided in Table 2 below. It was determined that the use of Kolliphor EL led to increased formation of impurities resulting from the hydrolysis of Compound 1.

TABLE 2
Hydrolytic Stability of Formulations of Compound 1

| | Hydrolytic Imp. | | | Hydrolytic Imp. | | |
|---|---|---|---|---|---|---|
| | 1% Area | | | 2% Area | | |
| Composition | T = 0 | 2 weeks | 4 weeks | T = 0 | 2 weeks | 4 weeks |
| 20% Crodamol, 40% Maisine CC, 40% | 0.09 | 0.16 | 0.2 | 0.28 | 0.3 | 0.33 |
| Capryol PGMC | | | | | | |
| 40% Maisine CC, 60% Capryol PGMC | 0.07 | 0.09 | 0.1 | 0.23 | 0.26 | 0.28 |
| 70% Labrasol ALF, 30% Capryol PGMC | 0.08 | 0.17 | 0.24 | 0.28 | 0.35 | 0.4 |
| 50% Labrasol ALF, 50% Kolliphor EL | 0.08 | 0.38 | 0.69 | 0.28 | 0.44 | 0.67 |
| 30% Labrasol ALF, 30% Kolliphor EL, | 0.1 | 0.3 | 0.6 | 0.3 | 0.42 | 0.72 |
| 40% Tween 80 | | | | | | |
| 70% Kolliphor EL, 30% Tween 80 | 0.08 | 0.51 | 0.87 | 0.28 | 0.5 | 0.82 |
| 40% Labrasol ALF, 40% Kolliphor EL, | 0.09 | 0.33 | 0.5 | 0.28 | 0.42 | 0.68 |
| 20% PEG 400 | | | | | | |
| 86% Polyethylene 6000, 10% Polyethylene | 0.22 | 1.02 | 2.32** | 0.31 | 0.42 | 2.32** |
| 1000, 1% Kolliphor P188, 0.15% Citric Acid | | | | | | |
| 60% Labrasol ALF; 40% Crodamol | 0.09 | 0.20 | 0.20 | 0.30 | 0.31 | 0.41 |
| 40% Miglyol 812N; 40% Crodamol | 0.08 | 0.35 | 0.65 | 0.25 | 0.40 | 0.51 |
| GMCC; 20% Span 80 | | | | | | |
| 40% Miglyol 812N; 40% Crodamol | 0.09 | 0.22 | 0.44 | 0.29 | 0.37 | 0.44 |
| GMCC; 10% Span 80; 10% Vit E TPGS | | | | | | |
| 80% Crodamol GMCC; 20% Span 80 | 0.08 | 0.26 | 0.44 | 0.26 | 0.36 | 0.45 |
| 40% Miglyol 812N; 40% Crodamol | 0.08 | 0.21 | 0.21 | 0.26 | 0.32 | 0.33 |
| GMCC; 20 Capryol 90 | | | | | | |
| 40% Crodamol GMCC; 60% Capryol 90 | 0.08 | 0.14 | 0.16 | 0.26 | 0.28 | 0.31 |
| 40% Maisine CC, 40% Capryol 90; 20% | 0.08 | 0.31 | 0.36 | 0.26 | 0.41 | 0.45 |
| Kolliphor RH40 | | | | | | |
| 40% Maisine CC; 40% Capryol 90; 20% | 0.07 | 0.09 | 0.09 | 0.24 | 0.26 | 0.28 |
| Vit E TPGS | | | | | | |
| 40% Crodamol GMCC; 40% Capryol 90; | 0.07 | 0.24 | 0.37 | 0.24 | 0.34 | 0.43 |
| 20% Kolliphor RH40 | | | | | | |
| 50% Labrasol ALF; 30% Crodamol | 0.08 | 0.15 | 0.18 | 0.27 | 0.29 | 0.31 |
| GMCC; 20% Tween 80 | | | | | | |
| 50% Labrasol ALF; 30% Crodamol | 0.08 | 0.22 | 0.19 | 0.26 | 0.32 | 0.32 |
| GMCC; 10% Tween 80; 10% Capryol 90 | | | | | | |
| 50% Labrasol ALF; 30% Crodamol | 0.08 | 0.15 | 0.36 | 0.28 | 0.29 | 0.40 |
| GMCC; 10% Tween 80; 10% Span 20 | | | | | | |
| 40% Labrasol ALF; 40% Crodamol | 0.09 | 0.25 | 0.43 | 0.29 | 0.34 | 0.43 |
| GMCC; 10% Tween 80; 10% Capryol 90 | | | | | | |
| 40% Labrasol ALF; 40% Crodamol | 0.09 | 0.23 | 0.25 | 0.3 | 0.35 | 0.36 |
| GMCC; 10% Tween 80; 10% Span 20 | | | | | | |
| 50% Labrasol ALF; 30% Crodamol GMCC; | 0.09 | 0.26 | 0.43 | 0.27 | 0.34 | 0.41 |
| 10% Kolliphor RH40; 10% Capryol 90 | | | | | | |
| 50% Labrasol ALF; 30% Crodamol GMCC; | 0.08 | 0.26 | 0.45 | 0.27 | 0.34 | 0.43 |
| 10% Kolliphor RH40; 10% Span 20 | | | | | | |
| 40% Labrasol ALF; 40% Crodamol GMCC; | 0.08 | 0.27 | 0.27 | 0.26 | 0.37 | 0.38 |
| 10% Kolliphor RH40; 10% Capryol 90 | | | | | | |
| 40% Labrasol ALF; 40% Crodamol GMCC; | 0.09 | 0.19 | 0.25 | 0.30 | 0.30 | 0.37 |
| 10% Kolliphor RH40; 10% Span 20 | | | | | | |
| 40% Maisine CC; 40% Capryol 90; 20% | 0.07 | 0.13 | 0.12 | 0.25 | 0.27 | 0.28 |
| Gelucire 44/14 | | | | | | |
| 40% Crodamol GMCC; 40% Capryol 90; | 0.07 | 0.15 | 0.27 | 0.26 | 0.28 | 0.35 |
| 20% Gelucire 44/14 | | | | | | |
| 70% Labrasol ALF, 30% Capryol 90 | 0.07 | 0.12 | 0.16 | 0.25 | 0.25 | 0.28 |
| 50% Labrasol ALF, 30% Crodamol | 0.07 | 0.23 | 0.16 | 0.25 | 0.31 | 0.31 |
| GMCC; 20% Capryol 90 | | | | | | |
| 40% Maisine CC; 60% Capryol 90 | 0.07 | 0.11 | 0.10 | 0.27 | 0.27 | 0.26 |

### Example 3: Preparation of Soft-Gels (Formulation A)

Fill Material Preparation: The fill material for Compound 1 5 mg softgels, was prepared in Becomix 2.5L (MV27) equipped with homogenizer and agitator blades. Potency adjustment was performed based on Compound 1 potency and the weight of Glyceryl monolinoleate, NF (Maisine® CC) was compensated. The concentration of BHT remained constant (0.2% of total fill weight). The active mixture was prepared by mixing Maisine® CC, Compound 1, and Butylated Hydroxytoluene (BHT) at 20° C.-30° C. for at least 2.5 hours until Compound 1 and BHT were dissolved. The product temperature was maintained at 20° C.-30° C. during mixing and de-aeration by adjusting the mixing vessel on cooling mode. The mix was de-aerated for 20 minutes, discharged into a clean and dry stainless-steel container and blanketed with nitrogen.

Gel Mass Preparation: One 200 kg gel melt of OET-004037 (L2DDXHBHM Gelatin Clear) which is composed of Gelatin Type 195 acid bone, NF, Sorbitol Special Glycerin Blend and purified water USP, EP was manufactured by gel preparation personnel. The gel melt was color converted to OET-005037@ 920P opaque white by the addition of titanium dioxide and purified water for use in the encapsulation of Compound 1 5 mg softgels.

Encapsulation: A 4 Oval die set and standard medicine pump was used for encapsulation. Immediately after encapsulation (filled and sealed), the fresh softgels were transferred along a conveyor into tumbler dryer baskets. The softgels were dried enough to withstand being spread onto shallow trays for tunnel drying without significant sticking or misshaping. Formulation A has the composition shown below in Table 3.

TABLE 3
5 mg Softgel Formulation A

| | Formulation A |
|---|---|
| Compound 1 (mg) | 5.000 |
| Maisine CC (mg) | 204.958 |
| BHT (mg) | 0.042 |
| Total weight per | 210.0 |
| sofgel (mg) | |

### Example 4: Preparation of 10 mg Soft-Gels

Preparation of 10 mg softgel: The fill material for Compound 1 10 mg softgels, was prepared according to the methods described above in Example 3. The various softgel preparations are provided in Table 4 below. Formulations A-E showed significantly improved hydrolytic stability of Compound 1 as compared to the present containing 86% Polyethylene 6000, 10% Polyethylene 1000, 1% Kolliphor P188, and 0.15% Citric Acid.

TABLE 4
10 mg Softgel Preparations

| | Formulation | Formulation | Formulation | Formulation | Formulation |
|---|---|---|---|---|---|
| | B | C | D | E | F |
| Compound 1 (mg) | 10.0 | 10.0 | 10.0 | 10.0 | 10.0 |
| Miglyol 812N | 64.0 | — | — | — | — |
| (mg) | | | | | |
| Crodamol GMCC | 64.0 | — | — | — | — |
| (mg) | | | | | |
| Caproyl 90 (mg) | 32.0 | 64.0 | 48.0 | 96.0 | — |
| Maisine CC (mg) | — | 64.0 | — | 64.0 | 199.96 |
| Vitamin E TGPS | — | 32.0 | — | — | — |
| (mg) | | | | | |
| Labrasol ALF | — | — | 112.0 | — | — |
| (mg) | | | | | |
| BHT (mg) | — | — | — | — | 0.04 |
| Total weight per | 170.0 | 170.0 | 170.0 | 170.0 | 210.0 |
| sofgel (mg) | | | | | |

### Example 5: Preparation of Additional 10 mg Softgels

Fill Material Preparation: The fill material for Compound 1 10 mg softgels, was prepared in Becomix 2.5L (MV27) equipped with homogenizer and agitator blades. Potency adjustment was performed based on Compound 1 potency and the weight of Miglyol 812N was compensated. The active mixture was prepared by mixing Miglyol 812N, Crodamol GMCC (pre-melted at 40° C.), Span 80, Vitamin E TPGS and Compound 1 at 20° C.-30° C. until Compound 1 is dissolved. The product temperature was maintained at 20° C.-30° C. during mixing and de-aeration by adjusting the mixing vessel on cooling mode. The mix was de-aerated for 20 minutes, discharged into a clean and dry stainless-steel container and blanketed with nitrogen.

Gel Mass Preparation: One 200 kg gel melt of OET-004037 (L2DDXHBHM Gelatin Clear) which is composed of Gelatin Type 195 acid bone, NF, Sorbitol Special Glycerin Blend and purified water USP, EP was manufactured by gel preparation personnel. The gel melt was color converted to OET-005037@ 920P opaque white by the addition of titanium dioxide and purified water for use in the encapsulation of Compound 1 10 mg softgels.

Encapsulation: A 4 Oval die set and standard medicine pump was used for encapsulation. Immediately after encapsulation (filled and sealed), the fresh softgels were transferred along a conveyor into tumbler dryer baskets. The softgels were dried enough to withstand being spread onto shallow trays for tunnel drying without significant sticking or misshaping. Formulation G has the composition shown below in Table 5.

TABLE 5
10 mg Softgel Formulation G

| | Formulation G |
|---|---|
| Compound 1 (mg) | 10.0 |
| Miglyol 812N (mg) | 58.0 |
| Crodamol GMCC (mg) | 68.0 |
| Span (mg) | 17.0 |
| Vitamin E TPGS | 17.0 |
| Total weight per sofgel | 170.0 |
| (mg) | |

### Example 6: Soft-Gel Storage Stability

Softgels were packaged in 40 count white round high density polyethylene 40 cc bottles with a white plastic child resistant cap and heat induction sealed with a 0.035″ white lined pulp board with foil, polyethylene terephthalate film and heat seal. placed on stability under ICH conditions of 5° C./ambient humidity and 25° C./60% RH. Additionally, stability studies for the formulations were conducted at accelerated conditions of 40° C./75% RH. Impurity levels were measured by HPLC. The stability results are provided in Table 6 below.

TABLE 6
Long Term Soft-gel Stability

| | | Total Impurities (% w/w) | | | | | | |
|---|---|---|---|---|---|---|---|---|
| | | | 1 | 3 | 6 | 9 | 12 | 24 |
| Formulation | Storage Condition | Initial | Month | Month | Month | Month | Month | Month |
| 10 mg LFC | 5° C./ambient | 3.3 | 3.5 | 3.3 | 3.4 | — | — | — |
| Campaign 1 | humidity | | | | | | | |
| 10 mg LFC | 5° C./ambient | 3.1 | NT | 3.3 | 3.2 | — | — | — |
| Campaign 2 | humidity | | | | | | | |
| 10 mg LFC | 25° C./60% RH | 3.3 | 4.0 | 5.3 | 7.3 | — | — | — |
| Campaign 1 | | | | | | | | |
| 10 mg LFC | 25° C./60% RH | 3.1 | NT | 5.0 | 6.5 | — | — | — |
| Campaign 2 | | | | | | | | |
| Formulation A | 25° C./60% RH | 3.7 | 3.6 | 3.4 | 3.2 | — | — | 3.1 |
| Formulation A | 40° C./75% RH | 3.7 | 4.0 | 4.0 | 4.1 | — | — | |
| Formulation F | 25° C./60% RH | 1.8 | 1.9 | 1.5 | 1.8 | 1.8 | — | — |
| Formulation F | 40° C./75% RH | 1.8 | 2.0 | 1.9 | 2.7 | — | — | — |

NT = Not Tested

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An oral dosage form comprising Compound 1 having the structure: 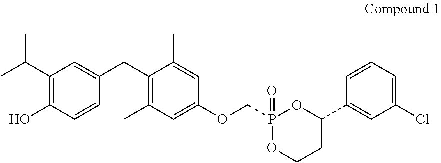
or a pharmaceutically acceptable salt thereof; and
one or more excipients selected from the group consisting of polyethylene glycol, caprylic acid triglyceride, capric acid triglyceride, caprylic acid diglycerides, capric acid diglycerides, caprylic acid monoglycerides, capric acid monoglycerides, polyethylene glycol sorbitan monooleate, oleic acid, polyethoxylated castor oil, lauroyl polyoxyl-32 glycerides, sorbitan monooleate, phosphatidylcholine, glyceryl stearate, oleaic acid, ascorbyl palmitate, lecithin, sorbitan monolaurate, tocophersolan, ethanol, propylene glycol monocaprylate, linoleic acid monoglyceride, oleic acid monoglyceride, linoleic acid diglyceride, Maisine® CC (glycerol/glyceryl monolinoleate), oleic acid diglyceride, linoleic acid triglyceride, oleic acid triglyceride, glycerol monocaprylocaprate, and combinations thereof.

2. The oral dosage form of claim 1, wherein Compound 1 and the one or more excipients are in a liquid fill within a exterior capsule.

3. The oral dosage form of claim 2, wherein the exterior capsule is a gelatin-based capsule

4. The oral dosage form of any one of claims 2-3, wherein the dosage form further comprises an antioxidant.

5. The oral dosage form of claim 4, wherein the antioxidant is butylated hydroxytoluene

6. The oral dosage form of any one of claims 2-5, wherein Compound 1 is present in the composition at a concentration of from 0.1 mg to 100 mg per gram of liquid fill.

7. The oral dosage form of a claim 6, wherein Compound 1 is present in the liquid fill at a concentration of from 5 mg to 25 mg per gram of liquid fill.

8. The oral dosage form of a claim 7, wherein Compound 1 is present in the liquid fill at a concentration of about 20 mg per gram of liquid fill.

9. The oral dosage form of any one of claims 2 to 8, wherein the excipient comprises Maisine® CC (glycerol/glyceryl monolinoleate).

10. The oral dosage form of claim 9, wherein Maisine® CC comprises 90% to 99% by weight of the liquid fill.

11. The oral dosage form of claim 10, wherein Maisine® CC comprises 93% to 96% by weight of the liquid fill.

12. The oral dosage form of claim 11, wherein Maisine® CC comprises 95% by weight of the liquid fill.

13. The oral dosage form of claim 9, wherein the excipient comprises 20% to 60% Maisine® CC by weight of the liquid fill.

14. The oral dosage form of any one of claims 2 to 13, wherein the liquid fill comprises Crodamol, Capryol PGMC, Labrasol ALF, or a combination thereof.

15. The oral dosage form of any one of claims 1 to 14, wherein the dosage form is characterized by having from 95% to 100% of the original amount of Compound 1 after 1 month of storage at 25° C. and 60% relative humidity (RH).

16. The oral dosage form of claim 15, wherein the dosage form is characterized by having from 98% to 100% of the original amount of Compound 1 after 1 month of storage at 25° C. and 60% relative humidity (RH).

17. The oral dosage form of claim 15, wherein the dosage form is characterized by having from 95% to 100% of the original amount of Compound 1 after 2 months of storage 25° C. and 60% relative humidity (RH).

18. The oral dosage form of claim 15, wherein the dosage form is characterized by having from 98% to 100% of the original amount of Compound 1 after 2 months of storage at 25° C. and 60% relative humidity (RH).

19. A method of preventing, treating, or ameliorating one or more fatty liver diseases in a subject in need thereof, comprising administering to said subject in need thereof the oral dosage forms of any one of claims 1 to 18.

20. The method of claim 19, wherein said fatty liver disease is selected from the group consisting of steatosis, non-alcoholic fatty liver disease, and non-alcoholic steatohepatitis.

21. The method of claim 19 or 20, wherein the method comprises administration of a second pharmaceutical agent.

22. The method of claim 21, wherein the second pharmaceutical agent is administered sequentially or simultaneously.

23. The method of any one of claims 19-22, wherein said method results in the prevention, treatment, or amelioration, of a fibrosis, fibrotic condition, or fibrotic symptom.

24. The method of any one of claims 19-23, wherein said method results in the reduction in the amount of extracellular matrix proteins present in one or more tissues of said subject.

25. The method of any of claims 19-24, wherein said method results in the reduction in the amount of collagen present in one or more tissues of said subject.

26. The method of claim 25 wherein said method results in the reduction in the amount of Type I, Type Ia, or Type III collagen present in one or more tissues of said subject.
